(19) United States
(12) Patent Application Publication
(10) Pub. No.: US 2022/0184029 A1
(43) Pub. Date: Jun. 16, 2022

(54) COMPOSITIONS AND METHODS FOR TREATING NEUROBLASTOMA

(71) Applicant: THE UNIVERSITY OF CHICAGO, Chicago, IL (US)

(72) Inventors: Lucy A. Godley, Chicago, IL (US); Anastasia Hains, Chicago, IL (US); Mark Applebaum, Chicago, IL (US); Susan Cohn, Chicago, IL (US)

(21) Appl. No.: 17/519,398

(22) Filed: Nov. 4, 2021

Related U.S. Application Data
(60) Provisional application No. 63/109,613, filed on Nov. 4, 2020.

(51) Int. Cl. A61K 31/395; A61K 31/472; A61K 31/69; A61K 38/15; A61K 31/436; A61K 31/675; A61K 31/565; A61K 38/12; A61K 31/277; A61P 35/00

(52) U.S. Cl. CPC A61K 31/395; A61K 31/472; A61K 31/69; A61K 38/15; A61P 35/00; A61K 31/675; A61K 31/565; A61K 38/12; A61K 31/277; A61K 31/436

(57) ABSTRACT

This disclosure relates to compositions and methods for treating a solid tumor, more specifically a neuroblastoma, in a subject in need thereof.

### CROSS-REFERENCE TO RELATED APPLICATIONS

This application claims the benefit of U.S. Provisional Application No. 63/109,613, filed Nov. 4, 2020, the disclosure of which is explicitly incorporated by reference herein.

### SEQUENCE LISTING

The instant application contains a Sequence Listing which has been submitted in ASCII format via EFS-Web and is hereby incorporated by reference in its entirety. Said ASCII copy, created on Jan. 21, 2022 is named 20-1702-US_Sequence-Listing.txt and is 4 kilobytes in size.

### BACKGROUND OF THE DISCLOSURE

### Field of the Invention

The present disclosure relates to compositions and methods for treating a solid tumor, more specifically a neuroblastoma, in a subject in need thereof.

### Technical Background

Ten-Eleven-Translocation 5-methylcytosine dioxygenases 1-3 (TET1-3) catalyze the conversion of 5-methylcytosine (5-mC) to 5-hydroxymethylcytosine (5-hmC), a modified cytosine base that helps to facilitate gene expression. TET1 is encoded by the TET1 gene and catalyzes 5-hmC production via oxidation of 5-mC in an oxygen, iron, and alpha-ketoglutarate dependent manner. The enzyme is highly expressed in embryonic stem cells owing to its importance as a transcriptional regulator. Consistent with this, elevated 5-hmC levels are associated with increased transcriptional activity leading to increased gene expression. Thus, TET1 is an important transcriptional regulator.

Hypoxia alters numerous cellular and physiological processes, including those associated with transcriptional regulation. For instance, in healthy cells, hypoxia decreases the catalytic activity of the oxygen-dependent dioxygenase TET1, while promoting increased accumulated of hypoxia inducible factor 1 alpha (HIF-1α). Hypoxia is also common in tumor cells, where a mismatch occurs in the rate of tumor cell growth and blood supply to support growth. The resultant hypoxic tumor microenvironments (TMEs) promote tumor cell migration and metastatic activity of the tumor.

The infant and young childhood cancer, neuroblastoma (NB), develops from immature nerve cells and is associated with a diverse array of clinical outcomes. NB-solid tumors arise from 5-hmC-rich neural crest tissue and have poorly organized vasculature that creates hypoxic regions within the tumor. The hypoxic regions induce TET1 transcriptional activation that allows for increased 5-hmC activities controlled by HIF-1α.

More specifically, hypoxic neuronal type MYCN-amplified NB cells exhibit increases in TET1 expression and global 5-hmC levels. The cells also demonstrate increased 5-hmC expression enriched along the gene bodies of hypoxia response genes in hypoxic conditions. These changes support continued growth and metastasis of NB tumors. Notably, both responses are controlled by HIF-1α.

Despite this understanding, effective treatment strategies in solid tumors, such as NB remain elusive. Indeed, NB diagnosis often occurs once the disease has spread, which increases the risk of elevated disease severity. Therefore, new therapeutic approaches that can effectively prevent or slow NB metastasis are needed.

### SUMMARY OF THE DISCLOSURE

This disclosure describes compositions and methods for treating solid tumor cancers including neuroblastoma.

As described below, in a first aspect the present disclosure provides a method for treating a neuroblastoma (NB) in a subject in need thereof, comprising obtaining a sample of an NB via tumor biopsy from the subject; measuring MYCN gene expression in the NB tumor sample; and administering a therapeutically effective amount of a CXCR4 antagonist to the patient if MYCN amplification level in the sample is elevated compared to a control sample.

In one embodiment of the first aspect, the sample is obtained via tumor biopsy. In another embodiment of the first aspect, the tumor biopsy is a bone marrow biopsy, an endoscopic biopsy, a fine-needle aspiration, a core needle biopsy, a vacuum-assisted biopsy, an image-guided biopsy, a shave biopsy, a punch biopsy, an incisional biopsy, an excisional biopsy, or a surgical biopsy. In one embodiment of the first aspect, MYCN gene amplification is measured using FISH. In one embodiment of the first aspect, the CXCR4 antagonist is plerixafor, a T140 analog, BL-8040, TN14003, MSX-122, TG-0054, FC122, FC131, AMD070, an AMD070 derivative, FC131, AMD3465, an AMD3465 analogue, WZ811, MSX122, NB325, NSC56612, KRH-3955, CTCE-9908, POL6326, or combinations thereof. In one embodiment of the first aspect, the CXCR4 antagonist is administered if MYCN gene amplification is 4-fold or more. In one embodiment of the first aspect, the neuroblastoma is an MYCN-amplified neuroblastoma.

In a second aspect the present disclosure provides a method for treating a neuroblastoma (NB) in a subject in need thereof, comprising obtaining a sample of a NB tumor from the subject; measuring CXCR4 gene expression in the NB tumor sample; and administering a therapeutically effective amount of a CXCR4 antagonist to the patient if the CXCR4 gene expression level in the sample is elevated compared to control.

In one embodiment of the first or second aspect an optional step is determination if the NB tumor is hypoxic.

In one embodiment of the second aspect, the CXCR4 antagonist is plerixafor, a T140 analog, BL-8040, TN14003, MSX-122, TG-0054, FC122, FC131, AMD070, an AMD070 derivative, FC131, AMD3465, an AMD3465 analogue, WZ811, MSX122, NB325, NSC56612, KRH-3955, CTCE-9908, POL6326, or combinations thereof. In another embodiment of the second aspect, the CXCR4 antagonist is plerixafor, a T140 analog, BL-8040, TN14003, MSX-122, TG-0054, FC122, FC131, AMD070, an AMD070 derivative, FC131, AMD3465, an AMD3465 analogue, WZ811, MSX122, NB325, NSC56612, KRH-3955, CTCE-9908, POL6326, or combinations thereof. In another embodiment of the second aspect, the CXCR4 antagonist reduces or prevents NB cell migration.

In a third aspect, the present disclosure provides a method for treating a solid tumor in a subject in need thereof, comprising reducing or preventing tumor cell migration by administering to the subject a therapeutically effective amount of a CXCR4 antagonist; and administering to the subject a therapeutically effective amount of a secondary therapeutic agent.

In one embodiment of the third aspect, the secondary therapeutic agent is a MYCN inhibitor that eliminates or reduces MYCN binding to the superenhancer located in first intron and/or the second intron of the solid tumor ten-eleven translocation methylcytosine dioxygenase 1 (TET1) gene.

In one embodiment of the third aspect, the secondary therapeutic agent is an antineoplastic agent, hypoxia-inducing factor-1α, hypoxia-inducing factor-1β inhibitor, an inhibitor that binds or reduces the superenhancer located in TET1 intron 1 (S1) an inhibitor that binds or reduces the superenhancer located in the second intron of TET1 (S2), or a MYCN inhibitor. In one embodiment of the third aspect, the secondary therapeutic agent is a hypoxia-inducible factor (HIF) inhibitor. In one embodiment of the third aspect, the HIF1 inhibitor is Roxadustat, Bortezomib, Romidespin, Temsirolimus, Perifosine, 2-methoxyestradiol, Echinomycin, Geldanamycin, 17-AAG, 17-DMAG, or MK-6482. In one embodiment of the third aspect, the HIF-1 inhibitor eliminates or reduces HIF-1α function in the solid tumor. In one embodiment of the third aspect, the CXCR4 antagonist and/or secondary therapeutic agent is administered to the subject orally and/or intravenously.

In a fourth aspect, the present disclosure provides a method of treating neuroblastoma (NB) in a subject in need thereof including a) obtaining a sample of an NB via tumor biopsy from the subject; b) determining cell surface CXCR4 expression level in the NB tumor sample; and c) administering to the subject a therapeutically effective amount of a CXCR4 antagonist to the patient if the cell surface CXCR 4 expression level is elevated compared to control.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure and are incorporated in and constitute a part of this specification. The drawings illustrate one or more embodiment(s) of the disclosure, and together with the description serve to explain the principles and operation of the disclosure.

FIG. 1A-FIG. 1F. Hypoxic 5-hmC gains are enriched in regions that are important for neuronal morphology, hypoxia adaptation, epigenetic regulation, and cell migration. (FIG. 1A) FPKM values of all 5-hmC peaks plotted over time exposed to hypoxia. Timepoints consist of 0, 6, 12, 24, 48, and 72 hours of hypoxia (n=415,416). (FIG. 1B) A subset of peaks from A that featured a positive log fold change from 0 to 72 hours. Shaded regions indicate high fold-change between two timepoints. Peaks are plotted the same as in FIG. 1A, 5-hmC FPKM over time (n=189,949). (FIG. 1C) Log2 enrichment (y-axis) of peaks that increase from 0 to 6 hours plotted by genomic element. Peaks that were classified as ‘early’ have an increase greater than 1.2-fold from 0 to 6 hours and remain stable throughout the rest of the time course. Genomic elements (x-axis) consist of promoters, 5′ untranslated region (5′ UTR), coding domain sequences (CDS), 3′ untranslated region (3′ UTR), introns, enhancers, HIF-1 binding regions, CpG islands, CpG shores, and intergenic regions (n=26,061). (FIG. 1D) Enrichment of peaks that increase from 24 to 48 hours by genomic element. Peaks that were classified as ‘late’ were stable from 0 to 24 hours and increased greater than 1.1-fold between 24 and 48 hours (n=56,190). (FIG. 1E) Genes associated with the enriched enhancers from C run through PANTHER (23) biological process statistical overrepresentation analysis. Biological process is shown on the y-axis and enrichment score is shown on the x-axis. (FIG. 1F) Genes associated with enriched CDS in 1D run through PANTHER (23) biological process statistical overrepresentation analysis. Biological process is shown on the y-axis and enrichment score is shown on the x-axis. *** represents P value of less than 2.2e-16 for all figures.

FIG. 2A-FIG. 2E. MYCN binds a superenhancer located in TET1 intron 1 (TET1-S1), the second intron of TET1 (TET1-S2), and a predicted upstream enhancer site. (FIG. 2A) FPKM values of all 5-hmC peaks plotted over time exposed to hypoxia. TET1 expression data from RNA-sequencing of 41 neuroblastoma cell lines (11) graphed by MYCN-amplified and non-amplified designations. (FIG. 2B) TET1 and MYCN expression data from RNA-sequencing of 41 neuroblastoma cell lines (11). Cell lines are plotted in order of increasing TET1 expression. TET1 expression data are plotted in squares and on the left Y-axis. MYCN expression data are plotted in filled circles on the right Y-axis. (FIG. 2C) TET1 and MYCN expression data from RNA-sequencing of 161 neuroblastoma tumors (12). Tumors are plotted in order of increasing TET1 expression. TET1 is plotted in black squares on the left, and MYCN is plotted in filled circles on the right. (FIG. 2D) MYCN ChIP sequencing data from SK-N-BE2-C, Kelly, and NGP cells (14) at locus 10q21. The region corresponding to the first peak, from left to right, is DNA2. The second peak is TET1-intron 1 (TET1-S1), and the last peak is TET1-intron 2 (TET1-S2). The next track (labeled ‘Enhancer’) displays the known superenhancer in TET1. The third track (labeled ‘Gene Ref’) represents the gene positions in hg19 build of the human genome. The lowest track (labeled ‘Enhancer prediction’) is generated from ‘enhanceratlas.org’. Arrows represent the direction of gene transcription. (FIG. 2E) Real time qPCR validation of MYCN ChIP-sequencing performed in SK-N-BE2 cells. From left to right, positive control (LARP1), negative control (neg TET1), DNA2, TET1-S1, TET1-S2 binding sites are plotted on the x-axis. The y-axis corresponds to the amount of DNA pulled down normalized to the amount of input DNA. ***, **, * represent p-values of p<0.05, p<0.01, and p<0.001, respectively.

FIG. 3A-FIG. 3D. There is no correlation between expression levels of MYCN with TET2 or TET3 in neuroblastoma cell lines. (FIG. 3A) TET2 and MYCN expression data from RNA-sequencing of 41 neuroblastoma cell lines. Cell lines are plotted in order of increasing TET2 expression with TET2 on the left Y-axis (black squares) and MYCN on the right Y-axis (filled circles). (FIG. 3B) TET2 expression data from 161 tumors are plotted in order of increasing TET2 expression. TET2 is represented by black squares, and MYCN is represented by filled circles. (FIG. 3C) TET3 and MYCN expression data from RNA-sequencing of 41 neuroblastoma cell lines. Cell lines are plotted in order of increasing TET3 expression with TET3 on the left Y-axis (black squares) and MYCN on the right Y-axis (filled circles). (FIG. 3D) TET3 expression data from 161 tumors are plotted in order of increasing TET3 expression. TET3 is represented by black squares, and MYCN is represented by filled circles.

FIG. 4A-FIG. 4F. MYCN is sufficient to induce TET1 and TET3 expression but not 5-hmC levels. (FIG. 4A) Real time qPCR quantification of MYCN in TET21/N cells over a 5-day time course. MYCN-induced TET21/N cells (right bar in each set). MYCN-uninduced TET21/N cells are plotted in black. MYCN expression in MYCN-induced TET21/N cells (right bar in each set) is normalized to MYCN-uninduced TET21/N cells (black) on respective days. P values were determined with one-tailed t-tests (n=3). (FIG. 4B) TET expression analyzed from RNA-sequencing data from both MYCN-induced (right bar in each set) and MYCN-uninduced (left bar in each set) TET21/N cells 4 days post induction. TET1, TET2, and TET3 are plotted along the x-axis. FPKM is plotted on the y-axis (n=3). (FIG. 4C) Real time qPCR data for TET1 expression 5 days post MYCN induction from MYCN-induced and MYCN-uninduced TET21/N cells. Relative TET1 expression (y-axis) in MYCN-induced cells (right bar in each set) is normalized to respective TET1 expression in MYCN-uninduced cells (left bar in each set). Each day is plotted on the x-axis. P values were determined with one-tailed t-tests (n=3). (FIG. 4D) TET3 expression determined by real time qPCR 5 days post MYCN induction from MYCN-induced and MYCN-uninduced TET21/N cells. Relative TET3 expression (y-axis) in MYCN-induced cells (right bar in each set) is normalized to respective TET3 expression in MYCN-uninduced cells (left bar in each set). Each day is plotted on the x-axis. P values were determined with one-tailed t-tests (n=3). ***, **, * represent p-values of p<0.05, p<0.01, and p<0.001 respectively. (FIG. 4E) TET1 and MYCN protein levels 4- and 5-days post MYCN induction, assayed by Western blot. (FIG. 4F) Quantitation of 5-hmC level with UHPLC-MS/MS performed on days 4 through 7 days post MYCN induction in MYCN-induced cells (right bar in each set and MYCN-uninduced cells (left bar in each set).

FIG. 5A-FIG. 5C. Deletion of MYCN binding site in DNA2 has no impact on TET1 expression. (FIG. 5A) Model of MYCN binding around 10q21.3. Shown, a predicted enhancer site in gene DNA2 upstream of TET1. (FIG. 5B) Sequences of the deletions generated by a gRNA targeting the MYCN binding motifs in the DNA2 gene (ΔDNA2). Sequence identifiers are as follows: Ref (SEQ ID NO: 1), ΔDNA2-S1.1 (SEQ ID NO: 2), and ΔDNA2-S1.2 (SEQ ID NO: 3). (FIG. 5C) Real time qPCR of TET1 mRNA across ΔDNA2 CRISPR-edited SK-N-BE2 cell lines (n=3). ***, **, * represent p-values of p<0.05, p<0.01, and p<0.001, respectively.

FIG. 6A-FIG. 6E. Binding of TET1-S1 by MYCN is important for TET1 and 5-hmC level regulation. (FIG. 6A) Model of MYCN binding around 10q21.3. The two sites are: a superenhancer in the first intron of TET1, and the second intron of TET1. (FIG. 6B) Sequences of the deletions generated by gRNAs targeting the MYCN binding motifs in the TET1-S1 site in TET1 (ΔTET1-S1), and the TET1-S2 site in TET1 (ΔTET1-S2). Sequence identifiers are as follows for: Column A-Ref (SEQ ID NO: 4), ΔTET1-S1.1 (SEQ ID NO: 5), and ΔTET1-S1.2 (SEQ ID NO: 6) and Column B-Ref (SEQ ID NO: 7), ΔTET1-S1.1 (SEQ ID NO: 8), and ΔTET1-S1.2 (SEQ ID NO: 9) (FIG. 6C) Real time qPCR of TET1 mRNA across ΔTET1-S1, and ΔTET1-S2 CRISPR-edited SK-N-BE2 cell lines (n=3). (FIG. 6D) Western blot for TET1 protein in ΔTET1-S1 (left panel) and ΔTET1-S2 (right panel). In normoxia, all TET1 isoforms are detected and quantified with protein loading normalized to PARP1. (FIG. 6E) Quantitation of global 5-hmC levels in cells that lack the MYCN binding sites in TET1 (ΔTET1-S1 and ΔTET1-S2) was measured by UHPLC-MS/MS. Percent 5-hmC is calculated relative to guanine. ***, **, * represent p-values of p<0.05, p<0.01, and p<0.001, respectively.

FIG. 7A-FIG. 7D. Loss of both TET1-S1 and TET1-S2 results in lowered TET1 expression. (FIG. 7A) Model of MYCN binding in the first and second introns of the TET1 gene. (FIG. 7B) Sequences of SK-N-BE2 cells that lack both site TET1-S1 and site TET1-S2 (ΔTET1-S1/2). Sequence identifiers are as follows for: Column A-Ref (SEQ ID NO: 10), ΔTET1-S1-S2.1 (SEQ ID NO: 11), and ΔTET1-S1-S2.2 (SEQ ID NO: 12) and Column B-Ref (SEQ ID NO: 13), ΔTET1-S1-S2.1 (SEQ ID NO: 14), and ΔTET1-S1-S2.2 (SEQ ID NO: 15) (FIG. 7C) Real time qPCR of TET1 mRNA in ΔTET1-S1/2.1 and ΔTET1-S1/2.2 CRISPR-edited SK-N-BE (2) cells (n=3). Western blot for TET1 protein in ΔTET1-S1/2 in normoxia is normalized to TOP1. (FIG. 7D) Quantitation of global 5-hmC levels in ΔTET1-S1/2 cells were measured via UHPLC-MS/MS. Percent 5-hmC is calculated relative to guanine. ***, **, * represent p-values of p<0.05, p<0.01, and p<0.001, respectively.

FIG. 8A-FIG. 8O. Deletion of TET1-S1 abrogates hypoxic transcription of TET1, but 5-hmC is still induced in hypoxia. (FIG. 8A) HIF-1α and HIF-1β ChIP-seq data from SK-N-BE2 cells at locus 10q21.3 are plotted with IGV. Binding sites from the region are aligned with Gene Ref from hg19. (FIG. 8B) Real time qPCR of MYCN ChIP in hypoxic SK-N-BE2 cells. Targets include ΔTET1-S1, ΔTET1-S2, and negative control (neg TET1) (n=3). (FIG. 8C) Real time qPCR quantification of TET1 transcription in hypoxia across clones that lack the MYCN/HIF-1 binding site in the superenhancer (ΔTET1-S1 clones) relative to normoxic parental SK-N-BE2 TET1 expression. P-values were determined with one-tailed t-tests (n=3). (FIG. 8D) Western blot of parental, ΔTET1-S1, and negative control cells will assay TET1 isoform protein level in normoxia and hypoxia (n=3). (FIG. 8E) Quantitation of global 5-hmC levels in ΔTET1-S1 cells in hypoxia was measured via UHPLC-MS/MS. Percent 5-hmC is calculated relative to guanine. (FIG. 8F) Real time qPCR quantification of TET1 transcription in hypoxia across clones that lack the MYCN/HIF-1 binding site in the second TET1 intron (ΔTET1-S2 clones) relative to normoxic parental SK-N-BE2 TET1 expression. P-values were determined with one-tailed t-tests (n=3). (FIG. 8G) Quantitation of global 5-hmC levels in ΔTET1-S2 cells was measured via UHPLC-MS/MS. Quantitation of global 5-hmC levels in ΔTET1-S2 cells was measured via UHPLC-MS/MS. Percent 5-hmC is calculated relative to guanine. (FIG. 8H) Quantitative PCR of TET1 expression in ΔS1, ΔS2, and ΔS1/2 cells exposed to hypoxia for 48 hours. (FIG. 8I) TET1 protein level in ΔS1, ΔS2, and ΔS1/2 cells in normoxia (N) and hypoxia (H). (FIG. 8J) Global 3-hmC levels in ΔS1 (top), ΔS2 (middle), and ΔS1/2 (bottom) cells in normoxia (left bar per cell type tested) and hypoxia (right bar per cell type tested) as measured by mass spectrometry. (FIG. 8K) Protein degradation assays with cycloheximide in normoxic and hypoxic parental SK-N-BE(2) cells. (FIG. 8L) Protein degradation assays with cycloheximide in normoxic and hypoxic parental SK-N-BE(2) cells in which HIF1A was deleted. (FIG. 8M) Half-life calculations in parental and HIF1A cells in normoxic (left bar per cell type tested) and hypoxic (right bar per cell type tested) conditions. (FIG. 8N) HIF-1 and TET1 co-Immunoprecipitation in normoxic (N) and hypoxic (H) SK-N-BE(2) cells. (FIG. 8O) Fold change of 5-hmC levels in ΔHIF1A and parental cells under normoxia and hypoxia. Bars are from left to right per time indicated: parental normoxia, parental hypoxia, ΔHIF1A normoxia, and ΔHIF1A hypoxia, respectively. ***, **, * represent p-values of p<0.05, p<0.01, and p<0.001, respectively.

FIG. 9A-FIG. 9C. Deletion of both binding sites within TET1 results in decreased TET1 hypoxia expression in hypoxia. (FIG. 9A) Model of MYCN/HIF-1 regulation of TET1 in normoxia and hypoxia. (FIG. 9B) Real time qPCR of TET1 mRNA in ΔTET1-S1/2.1 and ΔTET1-S1/2.2 CRISPR-edited SK-N-BE (2) cells in hypoxia (n=3). Western blot for TET1 protein in ΔTET1-S1/2 cells is evaluated in normoxia (n=3) and normalized to TOP1. (FIG. 9C) Quantitation of global 5-hmC levels in hypoxic ΔTET1-S1-S2 cells measured via UHPLC-MS/MS. Percent 5-hmC is calculated relative to guanine. ***, **, * represent p-values of p<0.05, p<0.01, and p<0.001, respectively.

FIG. 10A-FIG. 10I. Cells lacking TET1-S1 exhibit defective cell migration in the presence of HIF-1. (FIG. 10A) and (FIG. 10B) Wound healing assays with SK-N-BE2 cells that lack the MYCN/HIF-1 binding site in the TET1 superenhancer (ΔTET1-S1 clones) observed in the presence of FG-4592 (DMSO control, FIG. 10A). (FIG. 10C) Transwell migration assays were performed with ΔTET1-S1 clones in hypoxia (n=3). Percentages were calculated with respect to average number of parental SK-N-BE (2) cells. (FIG. 10D) Transwell migration assays were performed with ΔTET1-S1/2 clones in hypoxia (n=3). Percentages were calculated with respect to average number of parental SK-N-BE (2) cells. (FIG. 10E) hMe-SEAL data obtained at 0- and 48-hours hypoxia visualized along the CXCR4 gene at locus 2q22.1. (FIG. 10F) CXCR4 expression from RNA-seq data from multiple neuroblastoma cell lines. Expression measured in normoxia (red) and 48 hours hypoxia (blue). *** represents p-value of less than 0.001. (FIG. 10G) Real time qPCR of CXCR4 expression in SK-N-BE (2) cells lacking ΔTET1-S1 and ΔTET1-S1/2 in normoxia and hypoxia at 48 hours (n=3). *, **, *** represent p-values of less than 0.05, 0.01, and 0.001, respectively. (FIG. 10H) Transwell assays performed with parental SK-N-BE(2) cells and treated with 10 μg/mL plerixafor or DPBS in hypoxia (n=4). (FIG. 10I) RNA-seq CXCR4 expression (log2) in NBL-WN, SK-N-BE(2), LA1-55n, NBL-S, SH-SY5Y, LA1-55, NBL-WS, and SHEP cell lines with varying MYCN amplification statuses and MYCN protein levels in normoxia (left bar per cell type tested) and hypoxia (right bar per cell type tested). * represents p-value of less than 0.05.

FIG. 11. CXCR4 is a direct target of MYCN and HIF-1α and transcription factors. hMe-SEAL data and ChIP-seq data obtained at 0- and 48-hours hypoxia visualized along the CXCR4 gene at locus 2q22.1.

FIG. 12A-FIG. 12C. CXCR4 expression during hypoxia in cell lines lacking tet1 expression. (FIG. 12A) Real time qPCR of CXCR4 expression in SK-N-BE (2) cells lacking ΔTET1-S1 and ΔTET1-S1/2 in hypoxia at 48 hours. (FIG. 12B) Real time qPCR of CXCR4 expression in NBL-WN cells lacking ΔTET1-S1/2 in hypoxia at 48 hours. (FIG. 12C) Effects of CXCR4 on NB cell migration in hypoxic SK-N-BE(2) ΔS1/2 cells. * represents p-values of less than 0.05.

FIG. 13A-FIG. 13C. Plerixafor treatment inhibition of neuroblastoma cell migration under hypoxic conditions. (FIG. 13A) Cell migration in SK-N-BE (2) and NBL-WN cells without (−) and with (+) Plerixafor as percent of SK-N-BE (2) cells parental migration. (FIG. 13B) Percentage wound closure changes in SK-N-BE (2) cells over 48 hours of hypoxia in PBS-control and Plerixafor treated cells. (FIG. 13C) Percentage wound closure changes in NBL-WN cells over 48 hours of hypoxia in PBS-control and Plerixafor treated cells. *, ** represent p-values of less than 0.05, and 0.01, respectively.

FIG. 14A-FIG. 14E. CRISPR gene editing generated deletions of S1 and S2. (FIG. 14A) Model of MYCN binding around 10q21.3. The two sites are (1) S1, in a superenhancer (orange) in the first intron of TET1, and (2) S2 in the second intron of TET1. (FIG. 14B) Sanger Sequencing of deletions generated by gRNAs targeting the MYCN binding motifs in the S1 site in TET1 (ΔS1), and the S2 site in TET1 (ΔS2) in SK-N-BE(2) cells. All clones lacked the CpG that makes up the core of the E-box motif. (FIG. 14C) Sanger sequencing of deletions generated by gRNAs targeting both MYCN binding motifs at S1 and S2 in TET1 (ΔS1/2). (FIG. 14D) TET1 mRNA expression after deletion of S1, S2, and S1/2 as measured by real time qPCR of TET1 mRNA across ΔS1, ΔS2, and ΔS1/2 CRISPR-edited SK-N-BE(2) cell lines (n=3). P-values were determined with one-tailed t-tests between the value of interest and the parental value. ***, **, * represent p-values of p<0.05, p<0.01, and p<0.001 respectively. (FIG. 14E)

Western blot for TET1 protein (top panel, cropped at ˜280 kDa) in parental, ΔS1 (left bar graph), ΔS2 (middle bar graph), and ΔS1/2 (right bar graph). In normoxia, all TET1 at 280 kDa detected and quantified. TOP1 from the same blot is visualized below (bottom panel, cropped at ˜110 kDa).

FIG. 15. Mechanism of HIF-1 regulation of TET-3 in hypoxia in an erythropoietic system in which HIF-1 regulation of TET-3 occurs by binding two sites in a predicted enhancer region in the second intron.

FIG. 16A-FIG. 16E. S1 and S2 control TET1 expression in normoxia and hypoxia in NBL-WN cells. (FIG. 16A) Model of MYCN and HIF-1a binding around 10q21.3. (FIG. 16B) Sequences of the deletions generated by gRNAs targeting the MYCN binding motifs in the S1 site in TET1 (ΔS1), and the S2 site in TET1 (ΔS2) NBL-WN cells. (FIG. 16C) Real time qPCR quantification of TET1 transcription in normoxia (left bar per in each cell type tested) and hypoxia (right bar in each cell type tested) across clones that lack the MYCN/HIF-1 binding site in the superenhancer (ΔS1 clones), clones that lack the MYCN/HIF-1 binding site in the second TET1 intron (ΔS2 clones), and clones that lack both (ΔS1/2 clones), compared to parental NBL-WN TET1 expression. P-values were determined with one-tailed t-tests between the parental cell line and the cell line of interest (n=3). (FIG. 16D) Western blots for TET1 protein in ΔS1, ΔS2, and ΔS1/2 NBL-WN cells in normoxia (left blot in each cell type tested) and hypoxia (right blot in each cell type tested). TOP1 loading control in bottom panel. (FIG. 16E) Fold change of hypoxic (right bar per in each cell type tested) 5-hmC levels over normoxic (left bar per in each cell type tested) 5-hmC levels in parental, NTC, and ΔS1/2 cells. Percent 5-hmC measured via UHPLC-MS/MS and is calculated relative to guanine. ***, **, * represent p-values of p<0.05, p<0.01, and p<0.001, respectively.

FIG. 17A-FIG. 17B. 5-hmC targets CXCR4 and CRKL have induced expression in hypoxia. (FIG. 17A) ChIP-sequencing data at the CXCR4 gene at locus 2q22.1 visualized with IGVtools. In the top track, the Gene Ref track of the CXCR4 gene. Arrows represent the direction of gene transcription. MYCN ChIP sequencing data from Kelly, NGP, NB1643, COGN415, and LAN5 cells are below the Gene Ref track. (FIG. 17B) CXCR4 and CRKL expression analyzed from RNA-seq data from both normoxic (left bar per in each gene expression bar graph) and hypoxic (right bar per in each gene expression bar graph) samples.

### DETAILED DESCRIPTION

Provided herein are methods and compositions for treatment of solid tumors, such as neuroblastoma.

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As used herein, the singular forms “a,” “an,” and “the” include plural referents unless the context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to an amount of a therapeutic agent, such as a C-X-C Motif Chemokine Receptor 4 (CXCR4) antagonist, which when administered to a subject, is sufficient to effect treatment for a disease or disorder described herein, such as reducing tumor cell migration and/or metastasis. The amount of a compound which constitutes a “therapeutically effective amount”, or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of one or more symptoms of the disease or disorder.

“Subject” refers to a warm-blooded animal such as a mammal, preferably a human, which is afflicted with, or has the potential to be afflicted with one or more diseases and disorders described herein.

“Pharmaceutical composition” as used herein refers to a composition that includes one or more therapeutic agents disclosed herein, such as CXCR4 antagonist, a pharmaceutically acceptable carrier, a solvent, an adjuvant, and/or a diluent, or any combination thereof.

“Gene expression” as used herein refers to the process by which information in a gene is used to synthesize functional gene products, such as messenger RNA and/or one or more proteins. Methods are known by one of skill in the art to measure and/or detect changes in gene expression. Measuring gene expression includes any method capable of determining changes in expression of the gene of interest, for example, MYCN and/or CXCR4 expression. Quantitative methods for determining changes in gene expression are known in the art and include, but are not limited to real time PCR, quantitative PCR, northern blotting, microarray, and Quantitative Fluorescence In Situ Hybridization (QFISH).

“MYCN amplification” (MNA), as used herein, is defined as greater than a 4-fold increase in MYCN signal number compared to centromeric reference probe, as measured by, for example, fluorescence in situ hybridization (FISH). Similarly, MYCN copy number can be considered as wild type (less than 2 fold increase in MYCN signal); MYCN gain (2-4 fold increase); low-level MNA (5-10 fold increase); and high-level MNA (>10 fold increase).

“Protein expression” as used herein refers to the method and pathways by which proteins are produced, modified, and regulated in living organisms. Expression of specific proteins can be detected using techniques known in the art for detecting the expression of a protein of interest on the cell surface, or within a cell. This includes, but is not limited to western blotting, mass spectrometry, 2D gel analysis, and fluorescent microscopy.

CXCR4 (C-X-C chemokine receptor type-4), also known as fusin or cluster of differentiation 184 (CD184), is an alpha-chemokine receptor specific for stromal-derived-factor-1. CXCR4 is present in newly developing neurons during embryogenesis where it plays a role in neuronal guidance. CXCR4 antagonists block the binding of C-X-C motif chemokine 12 (CXCL12 or stromal cell-derived factor 1) and the resultant downstream effects (e.g., cell migration). Based on the present disclosure, it is believed that inhibitors of CXCR4 (e.g., agents that diminish or completely block CXCR4 function, also referred to as CXCR4 antagonists herein) can be effective for treating neuroblastoma by reducing tumor cell migration and reducing the incidence of tumor cell metastasis.

Non-limiting examples of CXCR4 antagonists contemplated for use in the present disclosure include the immunostimulant, plerixafor, T140 analogs, BL-8040 (previously BKT140), TN14003, MSX-122, TG-0054, cyclic-pentapeptide-based antagonists including but not limited to FC122 and FC131, tetrahydroquinolines-based antagonists, including but not limited to AMD070 and AMD070 derivatives, indole-based antagonists including but not limited to FC131, Para-xylyl-enediamine-based compounds including but not limited to AMD3465 and AMD3465 analogues WZ811, MSX122, guanidine-based Antagonists including, but not limited to NB325, quinoline derivatives, including but not limited to NSC56612, KRH-3955, CTCE-9908, and POL6326, and combinations thereof.

In view of the present disclosure, the methods and compositions described herein can be configured by the person of ordinary skill in the art to meet the desired need.

### Compositions

In some embodiments, pharmaceutical compositions contemplated herein include a therapeutically effective amount of one or more CXCR4 antagonists. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

CXCR4 antagonists of the present disclosure can be administered through a variety of routes and in various compositions. For example, compositions containing CXCR4 antagonists can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, one or more CXCR4 antagonists is administered orally, and the subject is dosed on a daily basis.

The therapeutic agents (also referred to as “compounds” herein) described herein (e.g., CXCR4 antagonists and secondary therapeutic agents), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Non-limiting examples of contemplated secondary therapeutic agents include one or more antineoplastic agents. In other embodiments, contemplated secondary therapeutic agents include hypoxia-inducing factor-1α and/or hypoxia-inducing factor-1β inhibitors (HIF-1 inhibitors), including but not limited to, Roxadustat, Bortezomib, Romidespin, Temsirolimus, Perifosine, 2-methoxyestradiol, Echinomycin, Geldanamycin, 17-AAG, 17-DMAG, and MK-6482.

Other therapies can include inhibitors that bind or reduce the superenhancer located in TET1 intron 1 (S1) and the second intron of TET1 (S2). Additional therapies can also include MYCN inhibitors that eliminate or reduce MYCN binding to the superenhancer located in the first or second intron of the TET1 gene.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of CXCR4 antagonists and secondary therapeutic agents can be in the range of from about 0.0001 mg/kg/day, about 0.001 mg/kg/day, or about 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 mL of a pharmaceutical composition having a concentration of a CXCR4 antagonist at about 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 1000 μM in a pharmaceutically acceptable carrier.

### Methods

In some embodiments, methods of treating cancer, such as neuroblastoma, in a subject in need thereof include administering to the subject a therapeutically effective amount of one or more CXCR4 antagonists and optionally a second therapy and/or secondary therapeutic agent. Contemplated treatable cancers can include metastatic (e.g., stage IV cancer) or pre-metastatic solid tumors (e.g., stage I, II, or III cancers).

In some embodiments, the therapeutic methods contemplated herein include administering to the subject a pharmaceutical composition to the subject orally and/or intravenously.

In some embodiments, the therapeutic methods contemplated herein include administering to the subject a pharmaceutical composition including both one or more CXCR4 antagonists and one or more secondary therapeutic agents. In other embodiments, the therapeutic methods include administering a first pharmaceutical composition including one or more CXCR4 antagonists and a second pharmaceutical composition including one or more secondary therapeutic agents.

In some embodiments, one or more CXCR4 antagonists can be administered in conjunction with another therapy or therapies for cancer (a second therapy or secondary therapeutic agent). In some embodiments, the CXCR4 antagonist is delivered concurrently with the other therapy or therapies, or administration can be in series (e.g., a CXCR4 antagonist is administered before or after a secondary therapeutic agent).

In some embodiments, a solid tumor biopsy is used to determine the MYCN amplification status of the solid tumor in a subject. A sample can be obtained via tumor biopsy by using one of numerous methods known in the art. The tumor biopsy methods contemplated herein, include, but are not limited to a bone marrow biopsy, endoscopic biopsy, fine-needle aspiration, core needle biopsy, vacuum-assisted biopsy, image-guided biopsy, shave biopsy, punch biopsy, incisional biopsy, excisional biopsy, or surgical biopsy.

The solid tumor can be further tested to determine if the solid tumor is hypoxic. In some embodiments, the CXCR4 antagonist can then be administered alone or with a secondary therapy to the subject when MYCN is amplified and/or when the tumor is hypoxic.

In some embodiments, cell surface expression of CXCR4 of a biopsied tumor can be compared to cell surface expression of CXCR4 in a non-solid tumor cell or NB cell line. In some embodiments, a CXCR4 antagonist can then be administered alone or with a secondary therapy to the subject when it is determined the CXCR4 expression levels warrant such treatment.

### Examples

The Examples that follow are illustrative of specific embodiments of the invention, and various uses thereof. They are set forth for explanatory purposes only and should not be construed as limiting the scope of the disclosure in any way.

### Introduction

With the discovery of TET catalytic activity (1,2), there have been great advances in understanding the role of 5-hmC in various biological processes. 5-hydroxymethylcytosine (5-hmC) functions as a stable and distinct epigenetic mark associated with open chromatin and active gene transcription (3-6). 5-hmC is generated from the oxidation of 5-methylcytosine (5-mC) by TET enzymes. TET enzymes are dependent on oxygen, iron, and α-ketoglutarate for their activity. In a hypoxic environment, low oxygen levels decrease the catalytic activity of oxygen-dependent enzymes, including the TETs(7).

To study the relationship between hypoxia and TET activity, neuroblastoma (NB) was used as a model system. NB arises from neural crest tissue, which is known to have high levels of 5-hmC (8). In addition, NB has diverse clinical outcomes, occasionally resolving spontaneously but also progressing despite intensive medical intervention. Finally, solid tumors, such as NB, often have hypoxic regions due to a lack of organized vasculature. For these reasons, NB is an ideal model system in which to study how epigenetic changes due to a hypoxic environment influence cancer phenotype.

It was previously hypothesized that levels of 5-hmC would decrease in hypoxic NB cells, as TET enzymes would be less active in a low oxygen environment. However, surprisingly, the inventors of the current disclosure found that NB cell lines that have a neuronal morphology (N-type cells) and are MYCN-amplified had higher levels of global 5-hmC levels along with increased TET1 expression after exposure to 48 hours of hypoxia (3). The increased 5-hmC was found to be enriched along the gene bodies of hypoxic response genes, thus stimulating their induction when the cells were exposed to hypoxia (3). When TET1 expression was silenced, induction of these genes still occurred, but their expression did not reach the elevated level it did when TET1 was present (3). In addition, there was no change in global 5-hmC level (3). Similarly, when a subunit of the hypoxia master regulator HIF-1, HIF-1α, was silenced, TET1 induction was abolished, and 5-hmC levels did not change when cells were exposed to hypoxia (3). This indicated that TET1 and the 5-hmC epigenetic landscape were under the control of transcription factor HIF-1.

However, unanswered questions concerning this model remained. It was not determined why this phenotype occurred only in N-type MYCN-amplified NB cells. Additionally, it was unknown if HIF-1 regulated TET1 through a direct or indirect mechanism. The current disclosure presents evidence that MYCN regulates TET1 directly in MYCN-amplified neuroblastoma cell lines and that this regulation is necessary for maintaining high baseline TET1 levels. Also disclosed is the mechanism through which HIF-1 regulates TET1 and how this regulation of hypoxic TET1 impacts the phenotype of hypoxic NB cells. Further disclosed is evidence of reduction in NB cell migration in hypoxic conditions when NB cells are treated with plerixafor.

### Materials and Methods

### Cell culture

Neuroblastoma cell lines (SK-N-BE(2) and NBL-WN) were cultured in RPMI with 10% FBS. Both cell lines are male. Normoxic culture was performed at 37° C. under atmospheric O₂ and 10% CO₂ in a humidified incubator. For hypoxic exposure, cells were incubated under 1% O₂ and 10% CO₂ in a humidified chamber. TET21/N cells maintained with 1 ug/mL doxycycline until MYCN induction was needed, in which doxycycline was removed.

### Tumor Xenograft Experiments

Athymic mice, female, 6-8 weeks old, were procured from The Jackson Laboratory (stock no: 002019). Five million SK-N-BE(2) cells were diluted in PBS and injected subcutaneously into the flank of each mouse. Tumor length and width was then measured every other day with calipers. Volume was calculated using the formula V=(I*w²)/2. Mice were followed for 90 days unless tumor reached terminal size (3 cm³).

### RNA Isolation and Quantitative PCR

Total RNA was extracted with RNAzol reagent (Sigma-Aldrich) according to the manufacturer's protocol. RNA was converted to cDNA with Life Technologies High-Capacity cDNA Reverse Transcription Kit. Quantitative PCR was done with Power SYBR Green PCR Master on Applied Biosystems Fast 7500 machines.

### Protein Extraction and Western Blotting

Protein extraction was performed via high salt fractionation. Nuclear extracts were separated on SDS-PAGE with 6% acrylamide gels. After overnight transfer to a PVDF membrane (Millipore), membranes were blocked in 5% milk in TBST for one hour at room temperature and then probed with primary antibody: either α-TET1 (Genetex, GT1462) overnight or α-TOP1 (abcam, ab109374) for one hour or α-MYCN (abcam, ab16898) for one hour. After primary antibody incubation, membranes were incubated with their respective species secondary antibodies (α-Rabbit IgG Millipore, α-Mouse IgG Cell Signaling Technology) for one hour at room temperature. Results were detected via film exposure with Western-lightning Plus-ECL (PerkinElmer) following the manufacturer's instructions.

### CRISPR-Cas9 Genome Editing

To perform genome editing, gRNAs were inserted into plasmid Lenticrispr v2 (Addgene plasmid #52961) following the Zhang lab protocol (9). Lentiviral transduction was performed following the standard protocol provided by Addgene. Single cell clones were then cultured in 96 well plates until confluent. To genotype each clone, DNA was extracted via phenol:chloroform method, then GoTaq (Promega) PCR was performed with primers targeting the region of the edited site (see primer table) and PCR product sequenced.

### Detection of 5-hmC and 5-mC by UHPLC-MS/MS

Genomic DNA was extracted from cell lines following phenol:chloroform isolation protocols. Genomic DNA was hydrolyzed to nucleosides and run on an Acquity UPLC Oligonucleotide BEH C18 Column (Waters 186003950). The column was attached to an Agilent 6460 Triple Quad MS-MS with 1290 UHPLC for MRM Quantitation.

### 5-hmC Selective Chemical Labeling

5-hmC selective chemical labeling (hMe-Seal) was performed with the protocol described in Song et al., 2011. Briefly, 20 μg of sonicated genomic DNA was labeled with UDP-6-N3-glucose then biotinylated using DMCO-S-S-PEG3-Biotin Conjugate (Click Chemistry Tools). The biotinylated DNA was affinity purified and sequenced.

### ChIP-qPCR and ChIP and hMe-SEAL Sequencing Analysis

Crosslinked DNA was sonicated with a Covaris S220 Sonolab 7.2 1.0. The protocol for precipitation of Protein-DNA complexes was modified from Roland Wenger's protocol. DNA was amplified in ChIP-qPCR or sequenced.

Sequenced reads were aligned to the hg19 genome with Burrows-Wheeler Aligner. Peaks were called with MACS2. HTSeq-count was used to count number of reads per peak, which was then converted to FPKM. Data from hMe-SEAL time course analysis can be found at DOI. Data from HIF-1α ChIP can be found at DOI. Publicly available datasets used in this study can be found at the following sources (10-14).

### Wound Healing and Transwell Assays

Cells were grown on a 96 well plate until confluent. The plate was scratched with an Essen Woundmaker and then placed in IncuCyte and photographed every 4 hours. Transwell assays were performed with cell culture inserts with an 8 μm pore size (Fisher Scientific). Cells were incubated in serum free media on Falcon cell culture inserts in a 24 well plate. After 6 hours, cells were fixed with methanol/formalin and stained with crystal violet. Cells were photographed and counted. When cells were treated with plerixafor, a concentration of 10 μg/mL was used, and control cells were treated with PBS.

### Statistical Analysis

Statistical significance was calculated using one tailed t-tests for most biological experiments between two groups. When more than one group was compared, a one-way ANOVA test was used. For large datasets, for which a normal distribution could not be assumed, a Wilcox test was used. P-values from all tests were considered significant at <than 0.05. Significance tests were carried out in R, GraphPad Prism, and Microsoft Excel. Graphs were generated in R and GraphPad Prism Statistical thresholds and exact values for n can be found in the figure legends.

### Results

### Example 1: Hypoxic 5-hmC Gains are Enriched in Regions that are Important for Neuronal Morphology, Hypoxia Adaptation, Epigenetic Regulation, and Cell Migration

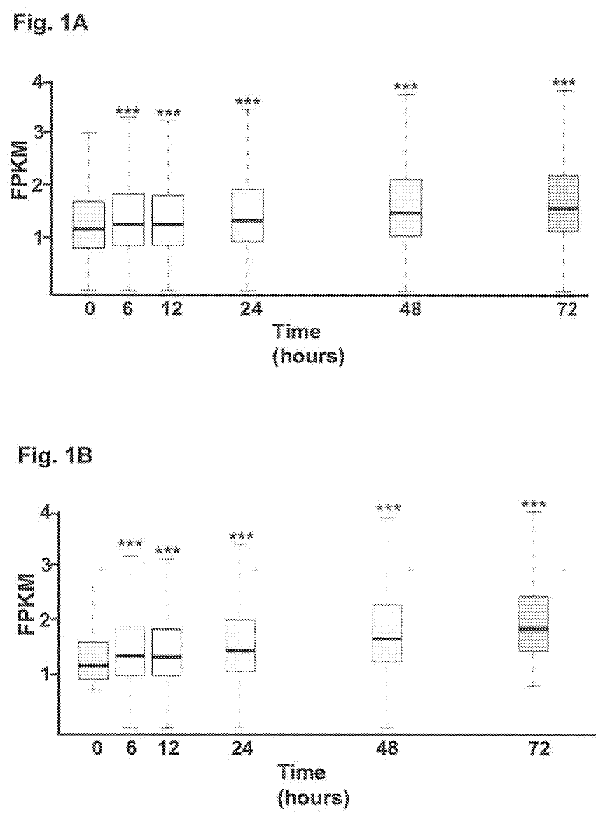

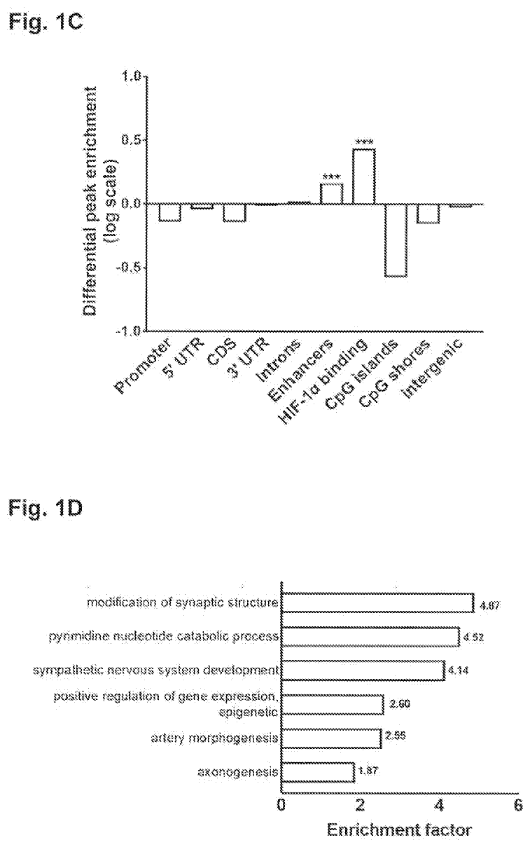

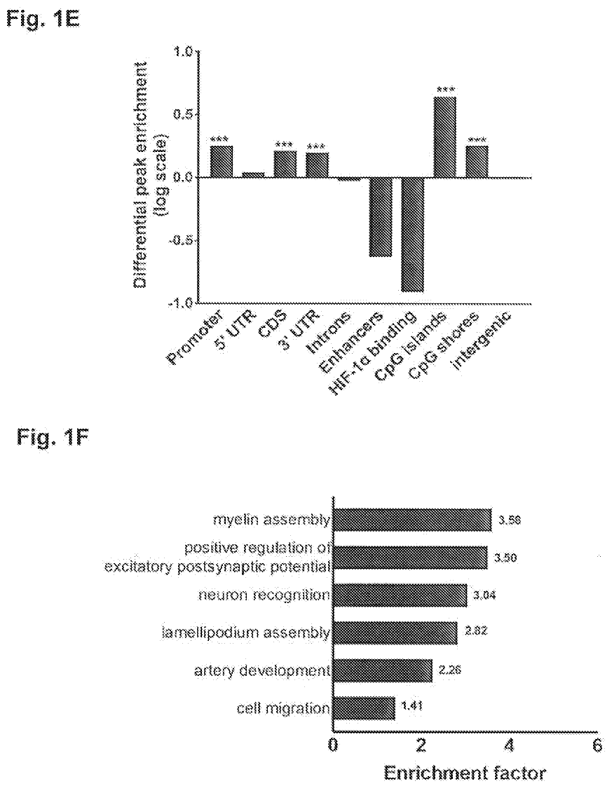

To examine how the 5-hmC epigenetic landscape changes in response to hypoxia, SK-N-BE(2) cells were subjected to hypoxic exposures of 0, 6, 12, 24, 48, and 72 hours. To determine 5-hmC enrichment at each of these time points, DNA was extracted and hMe-SEAL performed (15). First, all 5-hmC reads from all timepoints were converted to FPKM values and plotted over time (FIG. 1A). The mean FPKM of each time point increased over time, indicating the total amount of 5-hmC increased over time in hypoxia (FIG. 1A). To more closely examine these peaks that increase over time, peaks that did not have a higher FPKM value at 72 hours than at 0 hours were filtered out followed by replotting of the remaining peaks (FIG. 1B). It was clear that the highest rates of 5-hmC increase occurred bimodally: first, between 0 and 6 hours and second, between 24 and 48 (FIG. 1B). The peaks that increased between 0 and 6 hours were designated as ‘early,’ and the peaks that increase between 24 and 48 hours were designated as ‘late’. Next, it was determined if the 5-hmC peaks were being enriched in hypoxia and if early peaks were being enriched at different regions than late peaks. Enrichment analysis was performed with files containing the location and annotation of genomic elements and the amount of overlap with the 5-hmC peaks measured. Analysis of the early peaks found that early peaks were particularly enriched in enhancer and HIF-1α binding regions (FIG. 1C). Gene targets of the enriched enhancers were extracted in the early peaks and overrepresentation analysis was performed to determine what biological processes early 5-hmC enriched enhancers were associated with. Many of the biological processes that were overrepresented in the data set were related to neuronal morphology and development (FIG. 1D). Also overrepresented were processes that were downstream of the hypoxic response such as artery morphogenesis (FIG. 1D). These findings are expected considering it has been well established that these cells take on a more neural crest phenotype when exposed to hypoxia (16). More interestingly, epigenetic positive regulation of gene expression was also found to be an overrepresented biological process (FIG. 1D). Analysis of the late peaks showed that they were enriched in very different genomic regions than the early peaks. The late peaks were enriched in promoters, 5′ untranslated regions (5′ UTR), coding domain sequences (CDSs), 3′ UTR, CpG islands, and CpG shores (FIG. 1E). The late peak analysis was performed using the same overrepresentation analysis with the peaks associated with the CDS (FIG. 1F). Similar to the early peaks, many of these overrepresented targets were found to be part of the neuronal development and hypoxia biological processes. In contrast, epigenetic regulation of gene expression was not present and cell migration was identified as enriched. This implies that, although many of the early and late 5-hmC peaks are enriched in similar processes, there are unique targets as well. To investigate further, mechanisms by which the 5-hmC epigenetic landscape was controlled in normoxia and hypoxia and how aberration or loss resulted in a normoxic, or hypoxic phenotype were investigated.

### Example 2: MYCN Binds a Superenhancer Located in TET1 Intron 1 (S1), the Second Intron of TET1 (S2), and a Predicted Upstream Enhancer Site

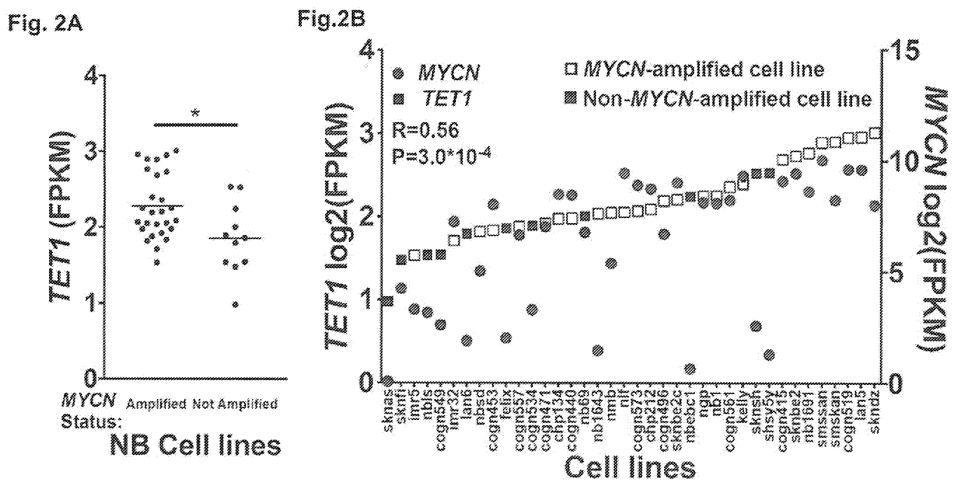

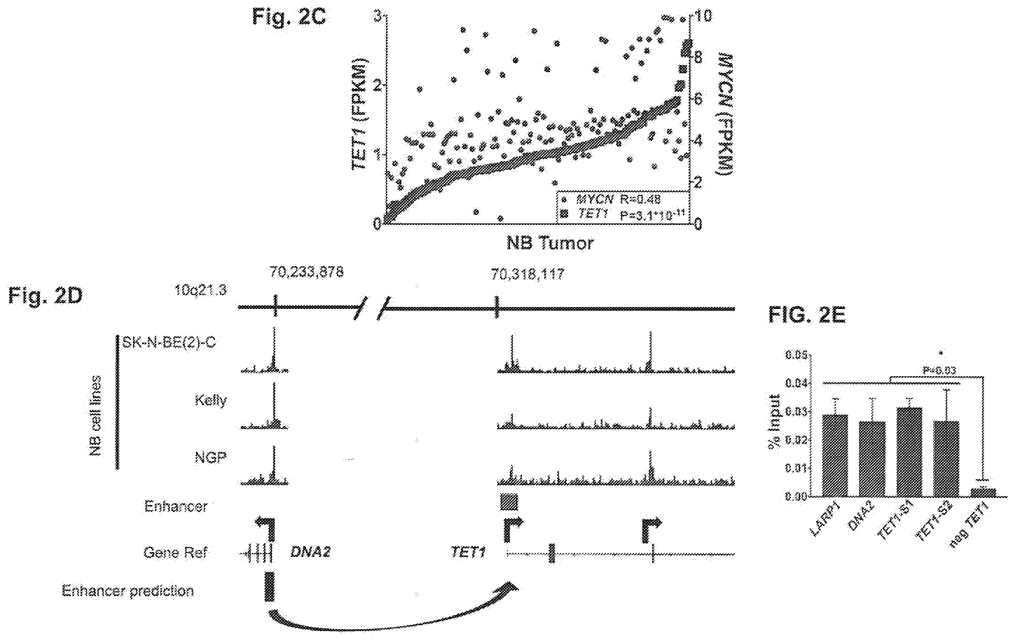

Using published RNA-seq data from NB cell lines and tumors (11,12), TET1 expression in MYCN amplified versus non-MYCN amplified NB cell lines (FIG. 2A) was graphed. MYCN amplified NB cells were found to have a higher baseline level of TET1 expression compared to non-MYCN amplified NB cells. Next, each NB cell line baseline TET1 expression was graphed and a positive correlation with MYCN expression (R=0.58, P=7.7e-5) (FIG. 2B) was shown. This is similar to a positive correlation between TET1 and MYCN expression in NB primary tumor (12) (FIG. 2C).

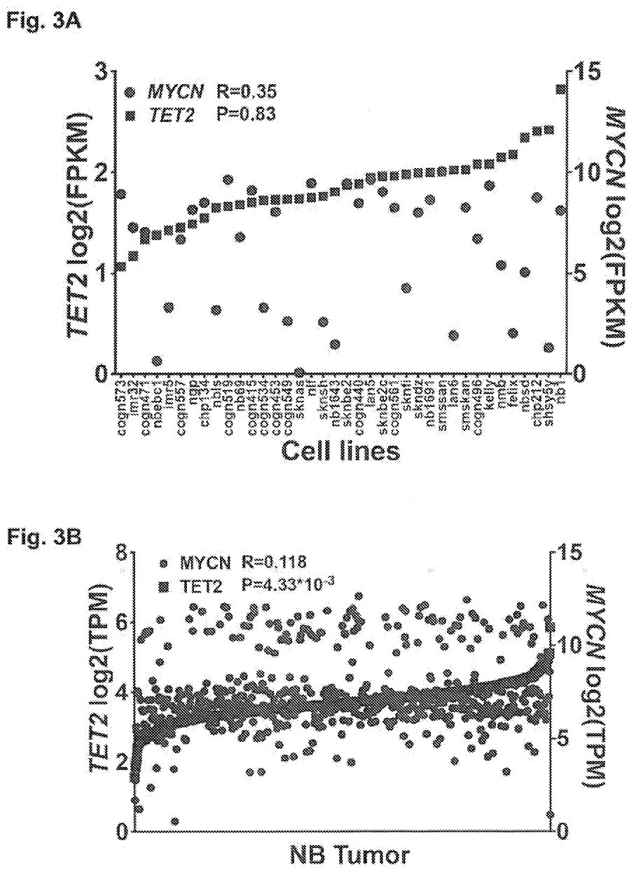

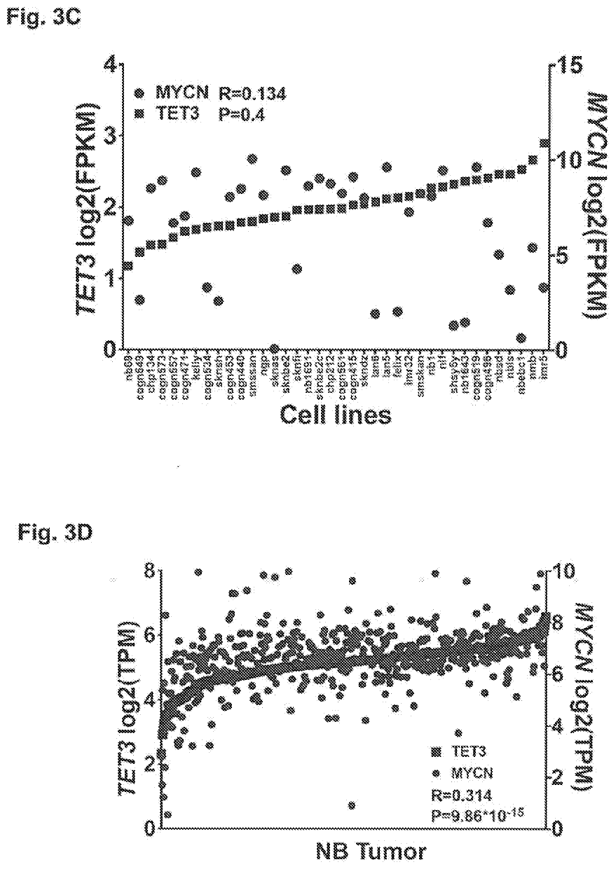

To confirm TET1 was the only TET enzyme gene that was positively correlated with MYCN expression, the same analysis was performed with TET2 and TET3 (11,12) (FIG. 3). Neither TET2 nor TET3 expression correlated with expression of MYCN in NB cell lines (FIGS. 3A and C). Analysis of TET2 and TET3 expression in tumors yielded the same result (12) (FIGS. 3B-3D). However, in NB tumors, there was a modest correlation between MYCN and TET3 (R=0.31, P=9.9e-15, FIG. 3D), but not between MYCN and TET2 (FIGS. 3B, 3D).

To determine if TET1 was regulated by MYCN through direct binding in MYCN amplified NB cell lines, publicly available ChIP-seq data at the TET1 locus was visualized (10,14). In order to determine if TET1 was a direct transcriptional target of MYCN, like HIF-1, that recognizes and binds an E-box element that consists of a canonical CANNTG sequence. Using HOMER (Heinz et al. 2010), numerous potential MYCN binding motifs were identified within and around TET1 (FIG. 2D), suggesting that MYCN could bind TET1 directly and regulate transcription from several binding sites. EnhancerAtlas was used to determine which of these sites is most likely to be bound by MYCN, and chromatin accessibility data, to visualize enhancer sites that could potentially regulate TET1 expression (FIG. 2D). Using data from the non-MYCN amplified cell line SK-N-SH, EnhancerAtlas identified two enhancers in TET1, in the first and second introns (FIG. 2D). In addition, the enhancer located in the first intron is encompassed by a superenhancer originally identified in an intergenic region in mice (28) (FIG. 2D). The UCSC LiftOver tool (29) was used to remap this mouse superenhancer to the hg19 human genome. Next, public MYCN ChIP-seq data was examined at the TET1 locus from all available MYCN-amplified NB cell lines to determine experimentally validated MYCN binding sites (FIG. 2D). These data showed direct binding of MYCN to two sites in TET1, referred to as Site 1 (S1) and Site 2 (S2) herein (FIG. 2D). The two binding sites have been described as associated with transcription of two different TET1 isoforms S1 is located centrally within both enhancers described in the first intron and S2 is located in the enhancer in the second intron of TET1 (FIG. 2D). The possibility that TET1 could be regulated by MYCN binding at distal enhancers was also investigated. EnhancerAtlas identified a potential TET1-associated distal enhancer located ˜88 kb upstream in the first intron of the gene DNA2 (FIG. 2D). Publicly available MYCN ChIP-seq data also revealed MYCN binding to this predicted enhancer site (FIG. 2D). MYCN binding to each of these three sites in SK-N-BE(2) NB cells was tested using MYCN ChIP-quantitative PCR (qPCR). Results indicated that all three sites were enriched with MYCN binding (FIG. 2E).

Herein, expression measurements of total TET1 are disclosed, including both isoforms. In addition, there was binding of a third site ˜88 kb upstream of TET1 (FIG. 2D). The third site is in the first intron of the gene DNA2, within a predicted TET1 enhancer site generated from data collected from SK-N-SH NB cell line (19). The binding of MYCN to these three sites was validated with MYCN ChIP-qPCR in SK-N-BE2 NB cells (FIG. 2E). It was hypothesized that one or more the MYCN binding sites played a role in regulating baseline expression of TET1. This suggests MYCN has the potential to regulate baseline TET1 through any or all of these three sites.

### Example 3: MYCN is Sufficient to Induce TET1 Expression but not 5-hmC Levels

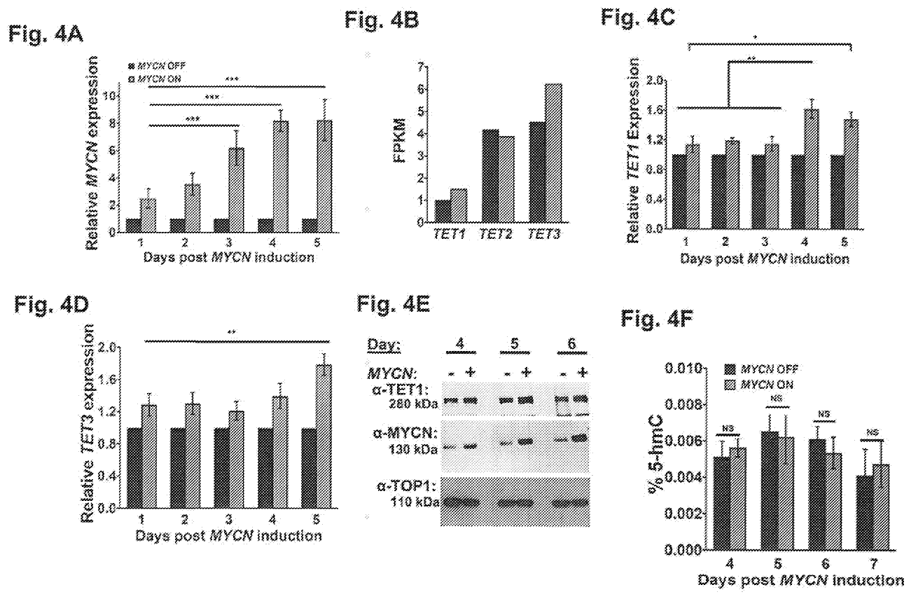

To establish if the presence of MYCN is sufficient to induce TET1 expression, the inducible-MYCN NB line SHEPTET21/N (TET21/N) was evaluated (20). Because TET21/N cells express MYCN through a tetracycline-off system, cells were first incubated with tetracycline for 24 hours. Tetracycline was removed and MYCN expression was induced. Over the course of 5 days RNA was extracted from cells with and without tetracycline each day. Real time qPCR measurements of MYCN expression were significantly induced 6-fold, three days after induction (FIG. 4A). Subsequent day 4 RNA-sequencing data from MYCN induced and non-MYCN induced cells demonstrated that TET1 and TET3 expression was elevated over cells than did not have induced MYCN (FIG. 4B). Real time qPCR confirmed TET1 expression was significantly increased 1.6-fold four days after MYCN induction (FIG. 4C). Western blotting of TET1 protein confirmed levels are elevated on day 4 and day 5 over non MYCN induced controls. Mass spectrometry was used to determine if induced expression of TET1 and TET3 enzymes resulted in increased total 5-hmC level, in order to quantify 5-hmC levels on days 4-7 post MYCN induction. There was no difference in 5-hmC level in MYCN induced cells compared to non-induced TET21/N cells (FIGS. 4E and 4F).

### Example 4: Loss of Both S1 and S2 in TET1 by MYCN Reduces TET1 Expression and 5-hmC Level Regulation

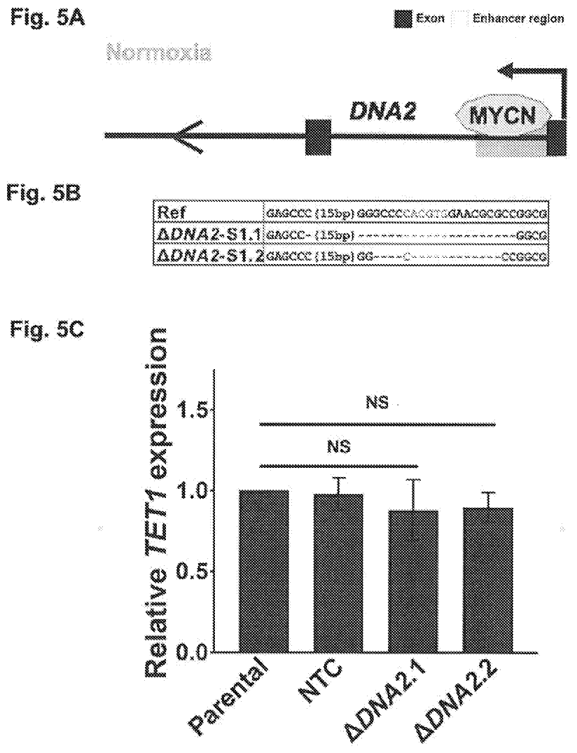

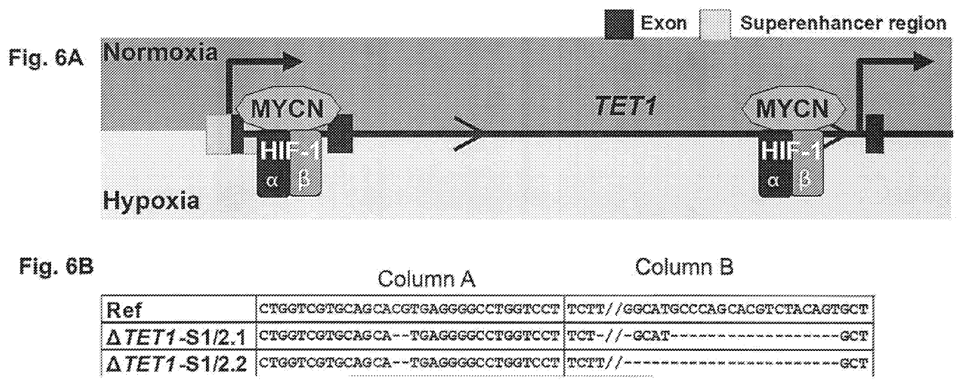

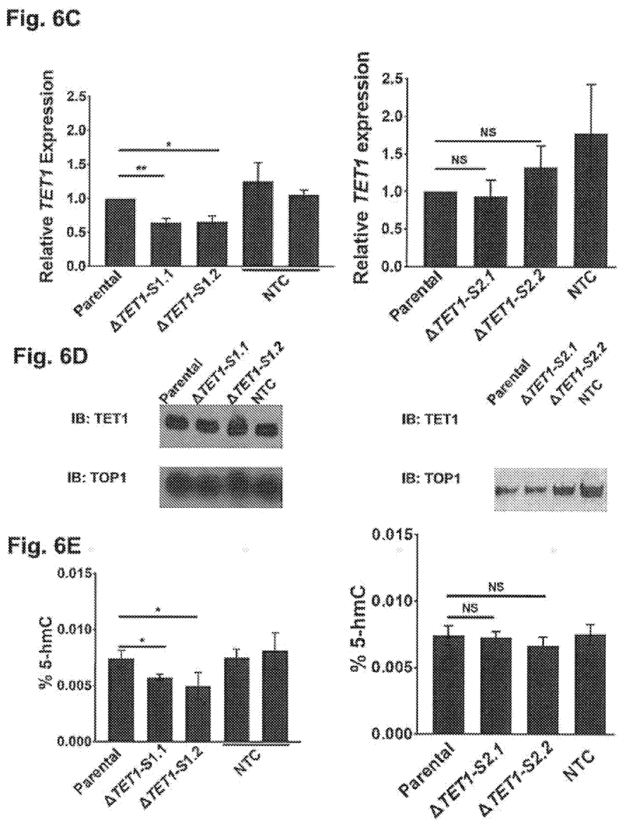

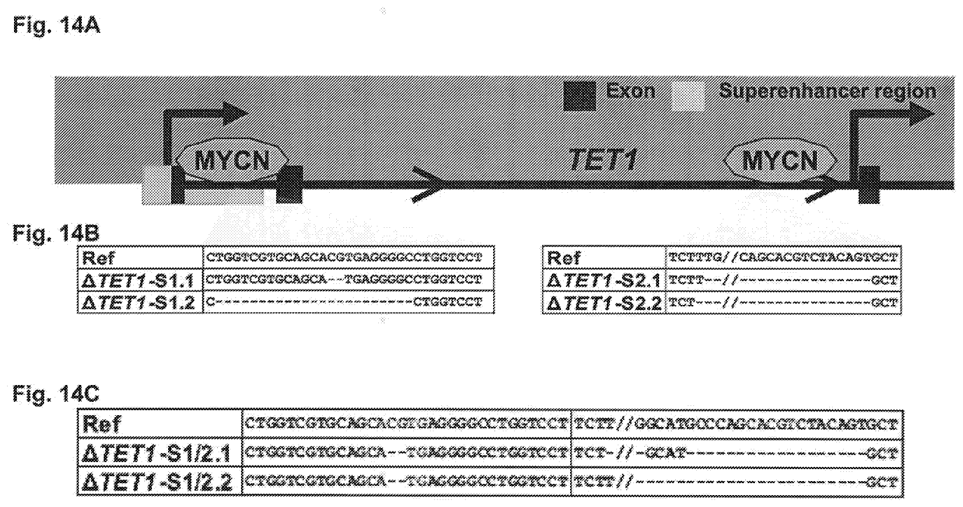

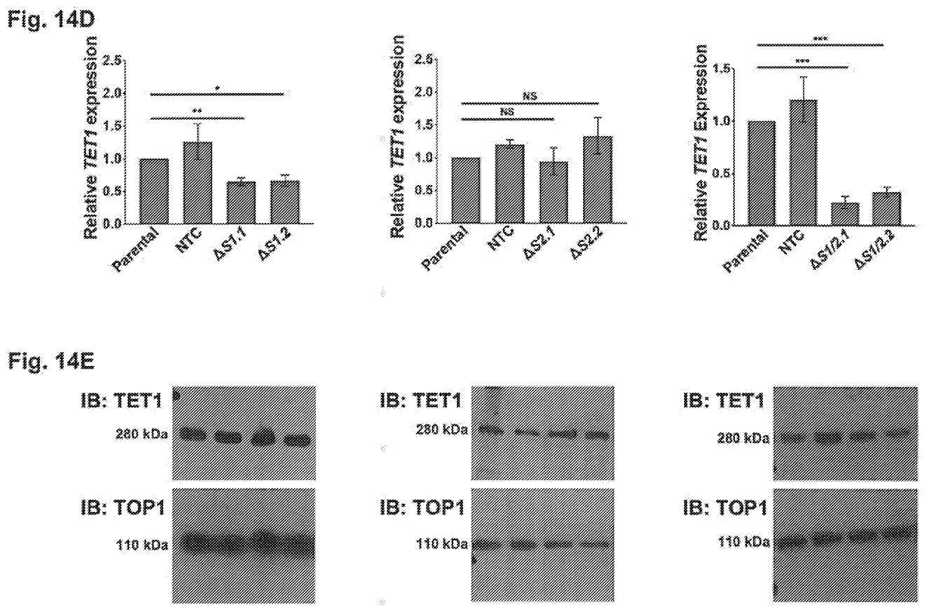

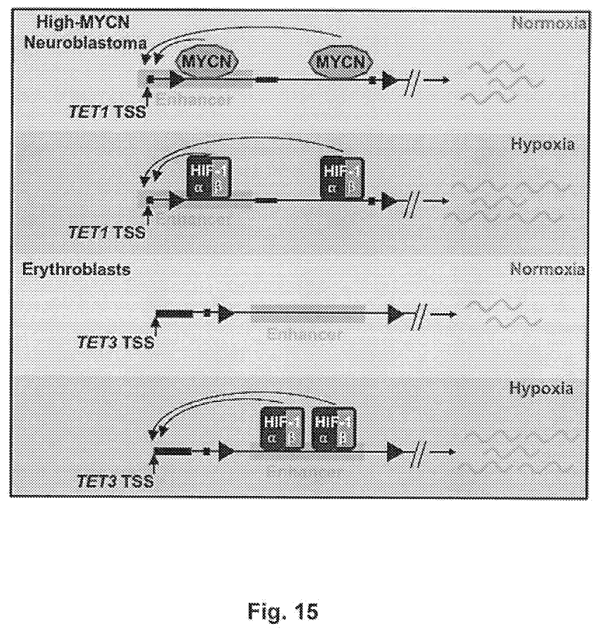

The ability of one or more of the MYCN binding sites in/near TET1 (FIGS. 1A and 14A) to directly regulate TET1 transcription was further investigated. To establish the functional role the binding sites, CRISPR-Cas9 gene editing was used to generate three lines that lack each of the three binding sites. Three gRNAs were designed to target the three sites that potentially regulate TET1 expression. The three cell lines generated from wild type SK-N-BE(2) cells were named: ΔDNA2, ΔS1, and ΔS2 (FIGS. 5A, 5B, and 6A) followed by “1” or “2” to distinguish individual clones targeting the same site (FIGS. 14A-14C). Double strand breaks were introduced at the E-box motif and delete each potential binding site, commonly on both alleles (FIG. 14B). From the mixed population of cells, we generated single cell clones and used PCR amplification and Sanger sequencing to confirm the binding site was eliminated (FIG. 14B). Deletions generated by this process were often unique in their placement and length, but all lacked the CpG that makes up the core of the E-box motif (FIG. 14B). Of these cell lines, two different clones were used that each featured a deletion of the MYCN binding site (FIGS. 5A-5B and FIG. 6A). RNA was extracted from each clone to measure TET1 expression. There was no significant difference between the TET1 expression of ΔDNA2, ΔS2, and normal parental SK-N-BE(2) cells (FIGS. 5C and 6C when either the potential binding site near DNA2 or S2 was deleted, suggesting that MYCN binding at these sites is not essential for maintaining baseline TET1 (FIGS. 4D and 5C). There was a slight, but highly reproducible, reduction of TET1 expression in ΔS1 cells (FIG. 6C).

5-hmC levels were also measured with mass spectrometry in normoxic ΔS1 and ΔS2 cells. ΔS1 cells had slightly reduced 5-hmC compared to control counterparts, reflecting the reduced expression of TET1 in these cells (FIG. 6E). ΔS2 cells had normal levels of normoxic 5-hmC compared to controls (FIG. 6E).

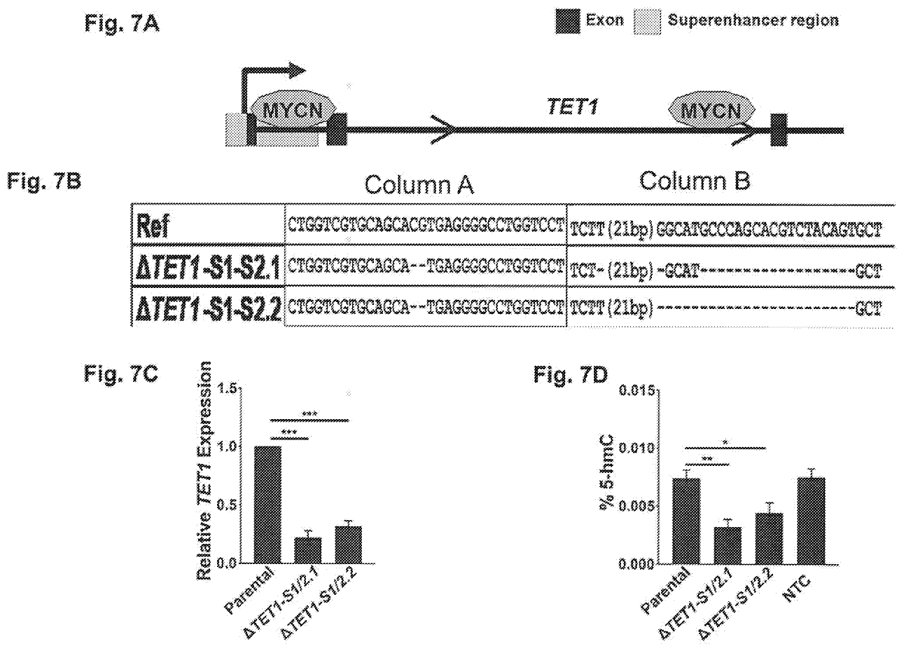

Because none of these sites fully abolished TET1 expression, a SK-N-BE(2) cell line was generated that lacked both of the binding sites in the TET1 gene (FIG. 7A). The clone, ΔS1/2, was generated from the ΔS1 cell line to have a different deletion at ΔS2 in each clone (FIG. 7B). When baseline TET1 expression was measured, it was found to be very low (FIG. 7C). More specifically ΔS1 and ΔS1/2 cells exhibited reductions in TET1 expression, of 0.7 fold and 0.25 fold respectively, compared to parental cells (FIG. 14D). This indicates S1 partially moderates baseline TET1, but both sites together maintain high baseline TET1 expression. However, TET1 protein levels in ΔS1 and ΔS1/2 cells remained comparable to parental cell line and NTC controls (FIG. 14E), indicating TET1 protein levels compensate for decreased TET1 mRNA and suggests that TET1 is relatively stable in these cells. Yet, when 5-hmC percentage was measured, it was revealed that these cells lacked about 50% of the parental control level (FIG. 7D).

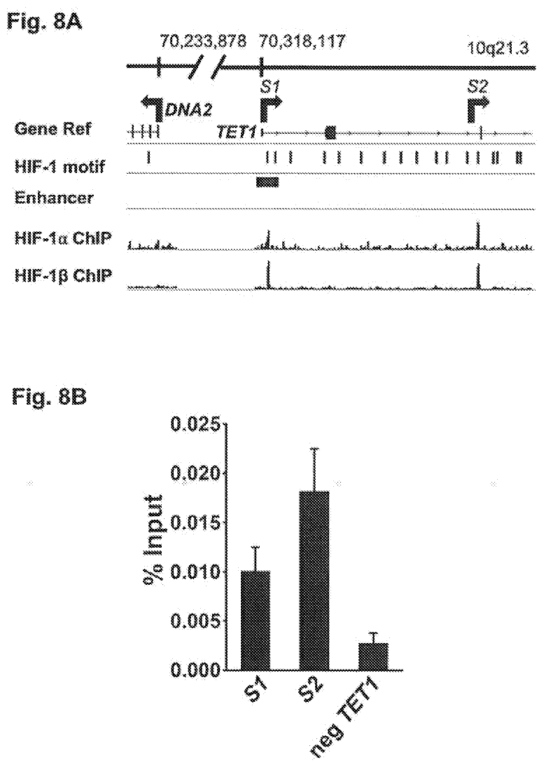

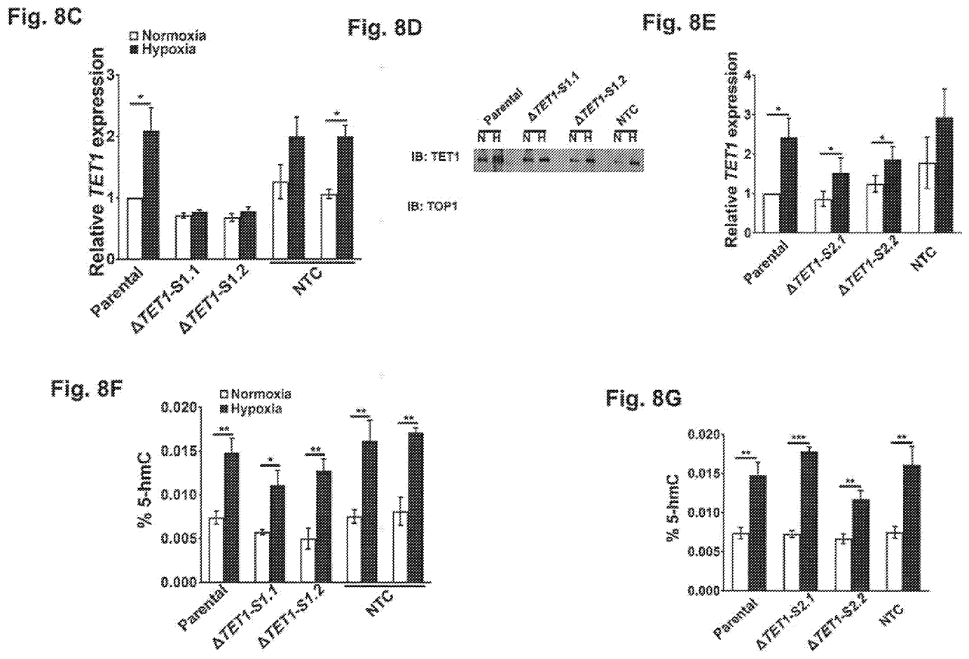

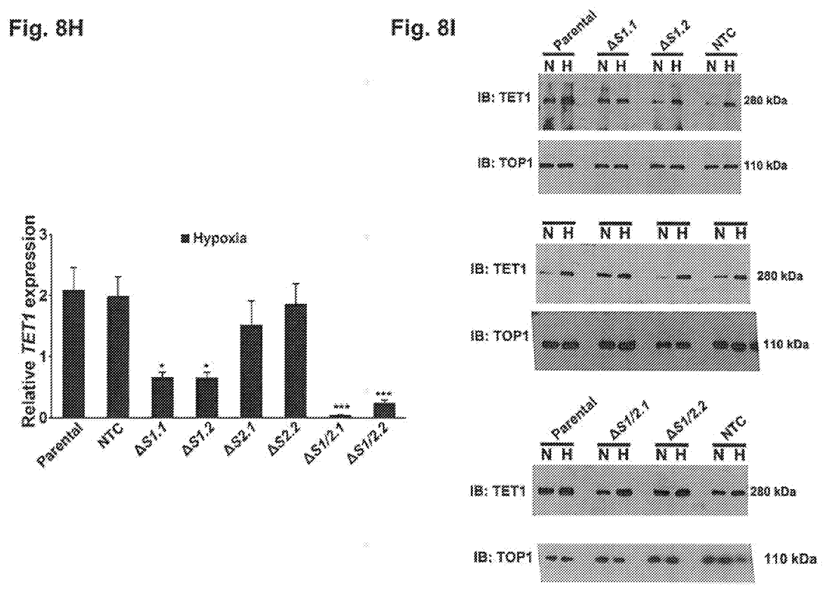

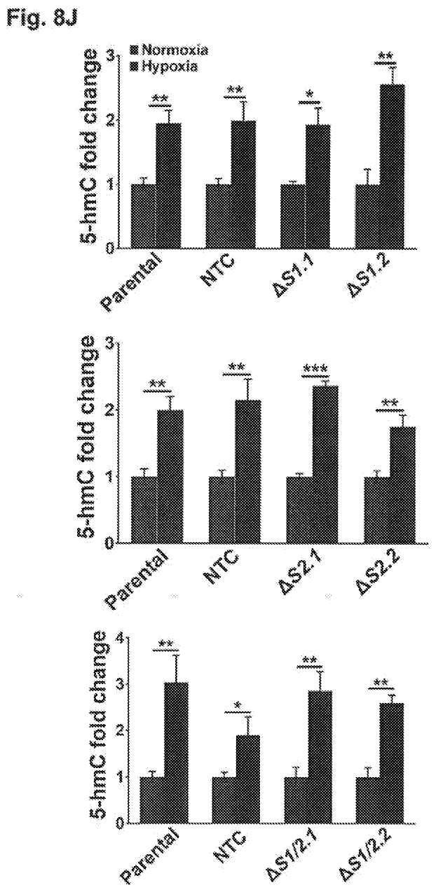

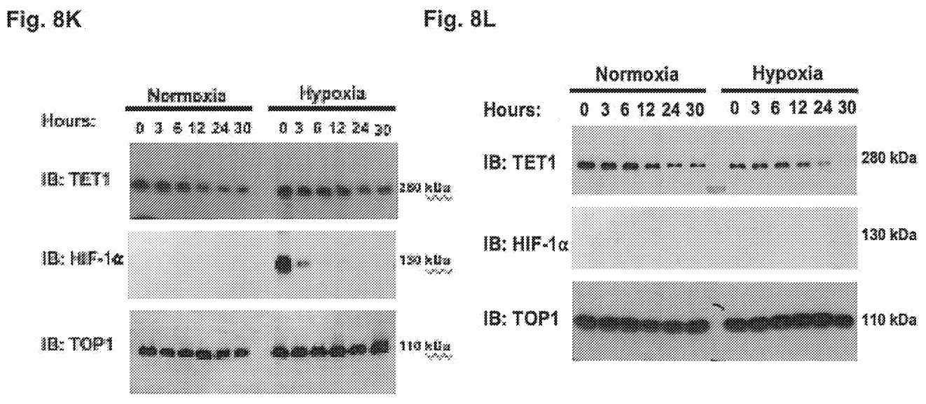

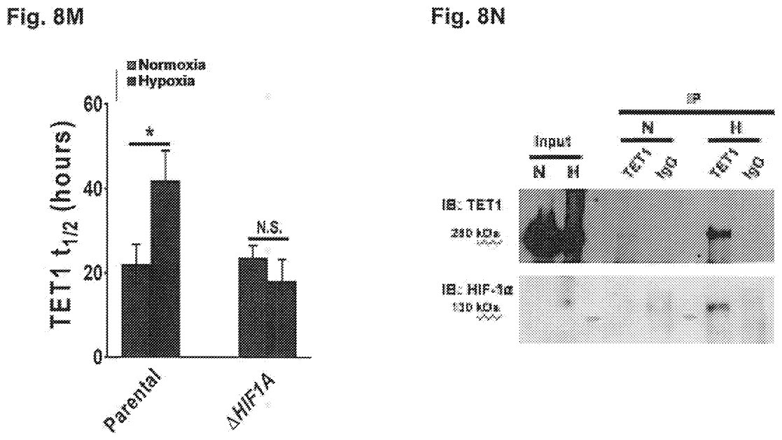

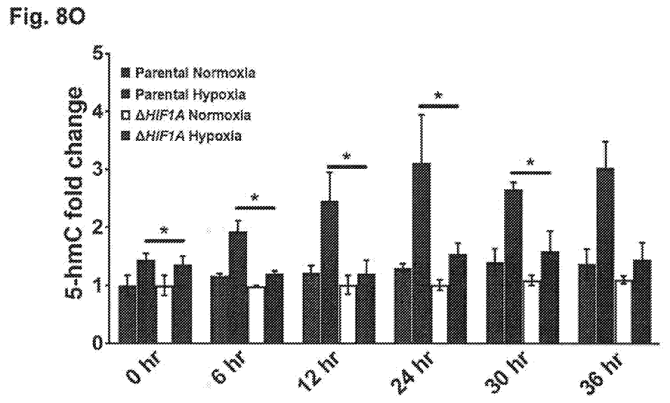

Example 5: Deletion of S1 abrogates hypoxic transcription of TET1 but 5-hmC is still induced in hypoxia TET1 expression has been shown to no longer be induced in hypoxia when HIF1A expression was targeted with siRNA (3). HIF-1α ChIP-sequencing was performed to further understand the mechanism of TET1 regulation in hypoxia (FIG. 8A). HIF-1α binds both S1 and S2 in TET1 but does not bind ΔDNA2. HIF-1β ChIP-sequencing data(13) was visualized to confirm that HIF-1β also binds S1 and S2 in TET1, but not at the binding motif in DNA2 that is bound by MYCN in normoxia (FIG. 8A).

Next, to determine if the presence of HIF-1α at S1 and S2 impacted the binding of MYCN to these two sites, MYCN ChIP-qPCR was performed to determine if HIF-1α still bound under these conditions. ChIP-qPCR showed that MYCN bound both S1 and S2 even in hypoxia, demonstrating it was not ousted by HIF-1 (FIG. 8B). Further experiments were undertaken to determine if the TET1 gene is regulated through the same sites in hypoxia as normoxia. Consistent with this, the gene edited cell lines were put under hypoxic conditions and TET expression measured. TET1 expression did not increase in hypoxia in ΔS1 cells (FIG. 8C). In contrast, TET1 expression was induced normally compared to controls in ΔS2 cells (FIG. 8F). TET1 in ΔS1, ΔS2, and ΔS1/2 cells that were exposed to hypoxia for 48 hours with qPCR (FIG. 8H). TET1 expression was low in hypoxia in ΔS1 cells compared to parental cells (FIG. 8H). However, TET1 protein level in ΔS1 cells was elevated in hypoxia over normoxia (FIG. 8I). Contrastingly, ΔS2 cells were no different from controls in TET1 expression or TET1 protein in hypoxia (FIGS. 8H-8I). However, hypoxic ΔS1/2 cells had very low TET1 compared to control lines (FIG. 8H), indicating hypoxic TET1 expression is exclusively mediated by S1 and S2 (FIGS. 6A-6B). Despite low TET1 mRNA, the protein was readily detectable (FIG. 8I), suggesting that the TET1 protein is stabilized in hypoxic conditions. 5-hmC levels were measured to determine the downstream impact of hypoxia (FIGS. 8F and 8G). Despite no TET1 induction, there was still an increase of 5-hmC in hypoxic ΔS1 cells (FIG. 8F). There was a similar increase in ΔS2 cells (FIG. 8G). Using mass spectrometry, we confirmed hypoxic 5-hmC levels were also elevated over normoxic 5-hmC levels in ΔS1, ΔS2, and ΔS1/2 cells (FIG. 8J).

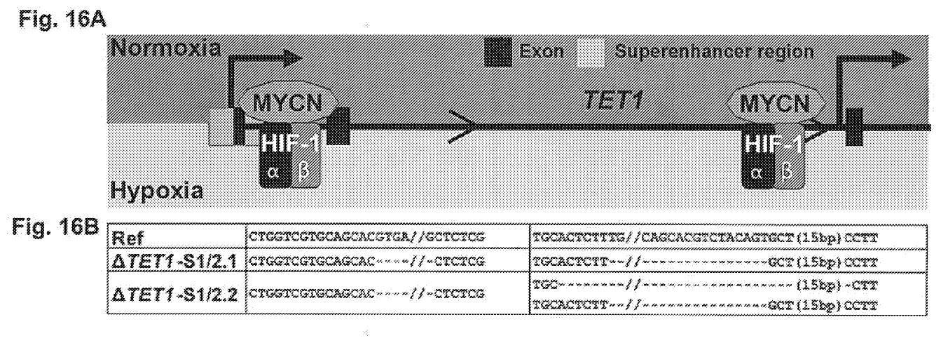

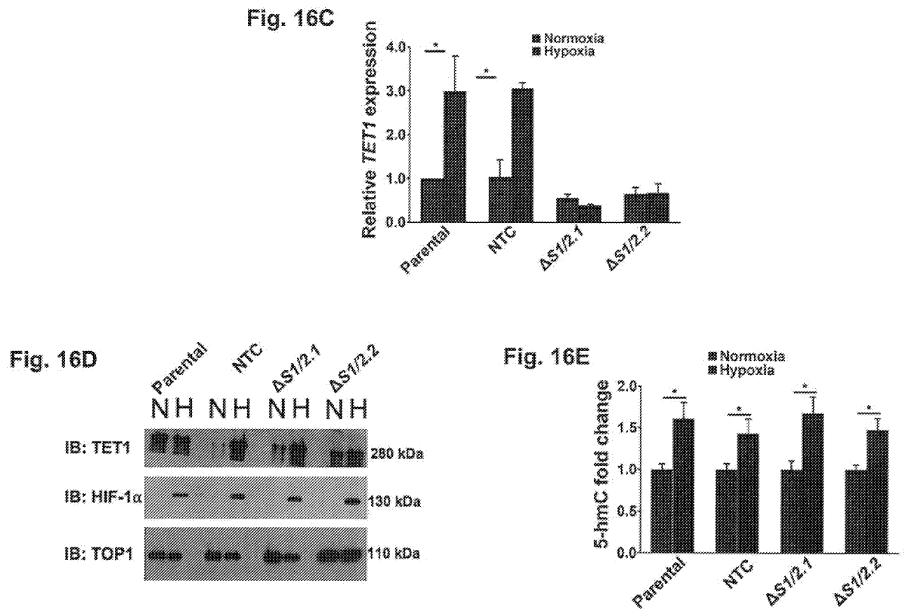

These findings were also determined in a second cell line, NBL-WN ΔS1/2 cells, to determine if the phenotype could be recapitulated (FIGS. 16A-16B). When both binding sites were deleted in the NBL-WN cell line, there was low TET1 expression in normoxia and hypoxia, similar to the SK-N-BE(2) ΔS1/2 cells (FIG. 16C). TET1 protein level was induced in hypoxia, regardless of the TET1 mRNA status, confirming TET1 protein status is similar across NBL-WN and SK-N-BE(2) ΔS1/2 cells (FIG. 16D). Similarly, global hypoxic 5-hmC levels were elevated in ΔS1/2 NBL-WN cells as well (FIG. 16E).

### Example 6: Deletion of Both Binding Sites within TET1 Results in Decreased TET1 Expression in Hypoxia

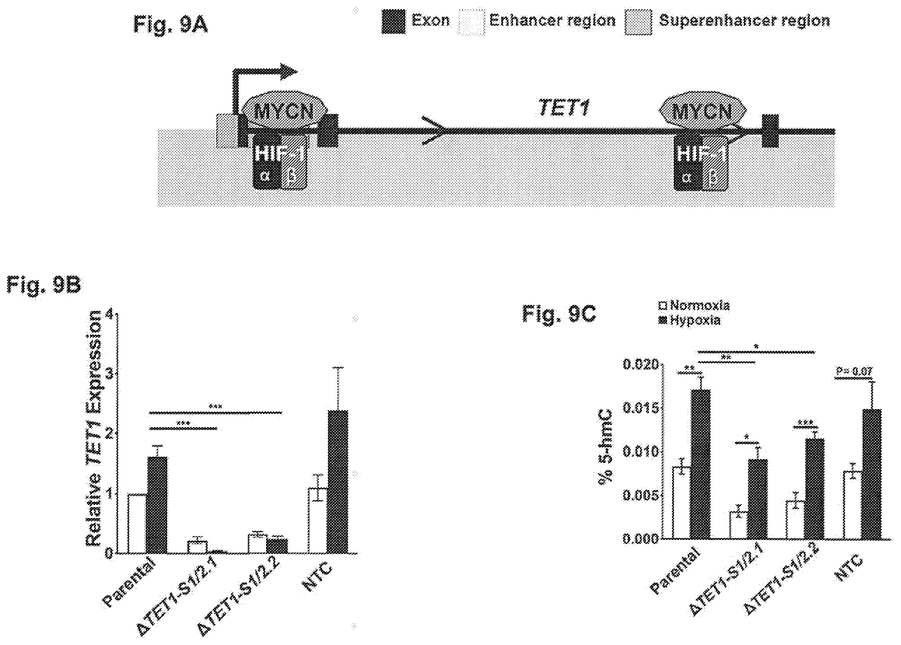

TET1 expression and the 5-hmC level were determined in cells that lack S1 and S2 under hypoxic conditions (FIGS. 9A-9B). When exposed to hypoxia, TET1 expression in ΔS1/2 cells decreases compared to normoxia. However, TET1 protein was detected (FIG. 9C). This indicates that TET1 activity is not only regulated at the transcriptional level but also at the protein level. The level of hypoxic 5-hmC in ΔS1/2 cells was determined in additional experiments. Although significantly attenuated, 5-hmC levels were still increased in hypoxia in ΔS1/2 cells (FIG. 9C).

### Example 7: Cells Lacking TET1-S1 Exhibit Defective Cell Migration in the Presence of HIF-1α

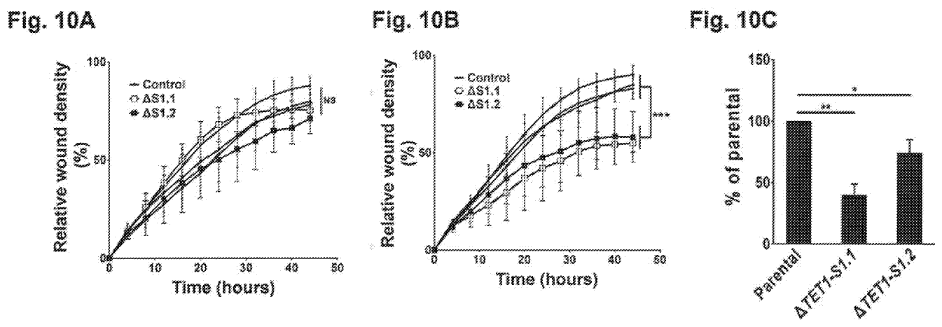

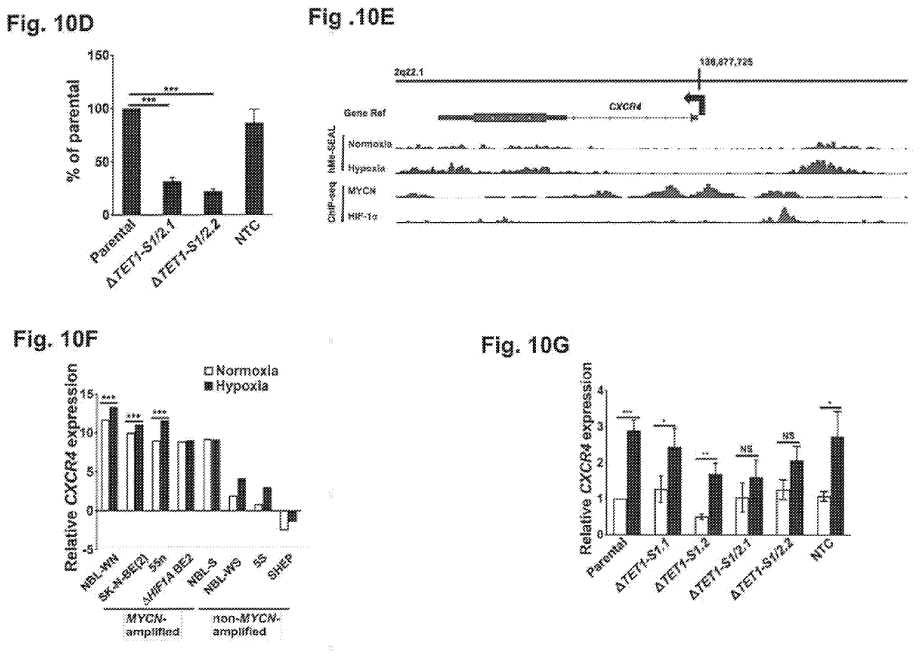

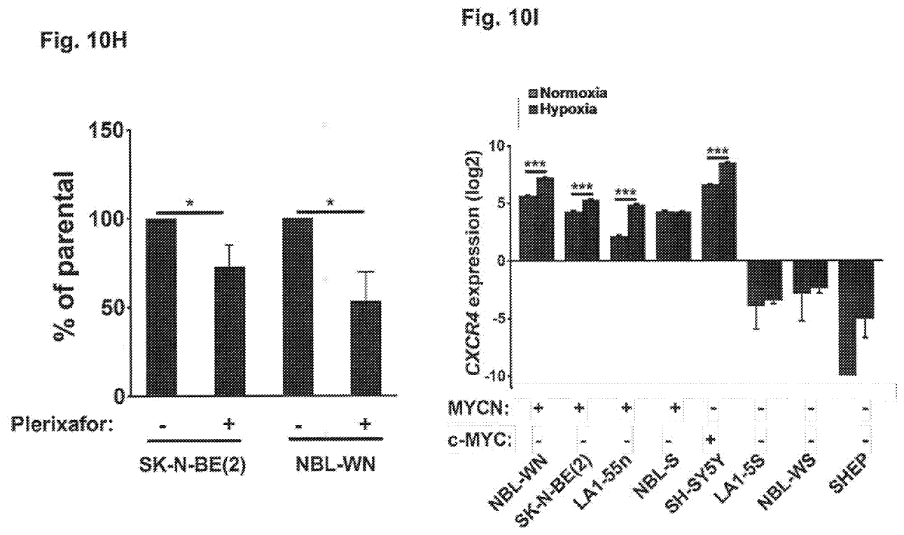

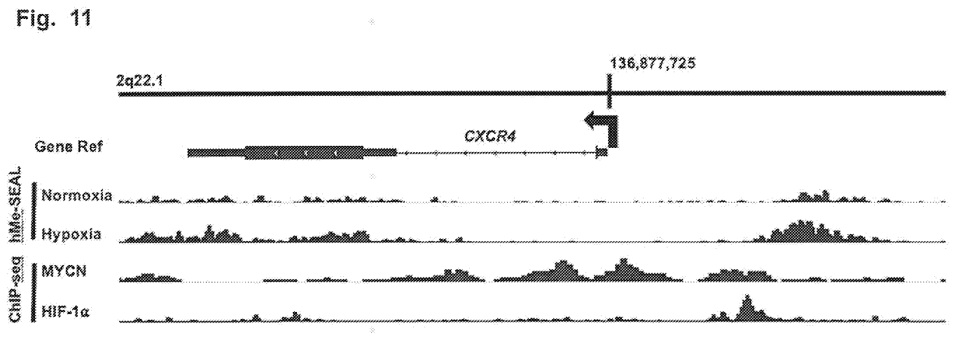

Although the ΔS1, ΔS2, and ΔS1/2 cells featured normal appearances under the microscope, a number of assays were performed to determine if there were any measurable phenotypes. Because many of these assays could not physically be performed in hypoxic conditions, a prolyl hydroxylase inhibitor (iPH) was utilized to allow HIF-1α to accumulate and induce a pseudo-hypoxic state. Assays measuring growth and cell cycle determined that ΔS1, ΔS2, and ΔS1/2 cells were normal in their growth and cell cycle stages. However, migration assays demonstrated phenotypic changes in some of the cells (see FIGS. 10A-10C). An initial scratch assay was performed using the ΔS1 cell line. Following incubation with iPH of DMSO for 24 hours, plates were scratched, and images of wound healing were taken every four hours for 48 hours. Although cells all migrated at the same rate in DMSO (FIG. 10A), ΔS1 cells migrated slower compared to the control lines in the presence of HIF-1Δ (FIG. 10B). To ensure this was a phenotype that occurred in a true hypoxic environment, migration was further measured using transwell assays. Cells were exposed to a hypoxic environment for 24 hours before being added to transwells. Results confirmed that cells lacking S1 migrated slower compared to their control counterparts in hypoxia (FIG. 10C). The migration of cells was measured in cells lacking both S1 and S2. Similar to the ΔS1 cells, these cells too migrated slower than control cells in hypoxia (FIG. 10D).

### Example 8: Molecular Mechanisms of Slow Migration

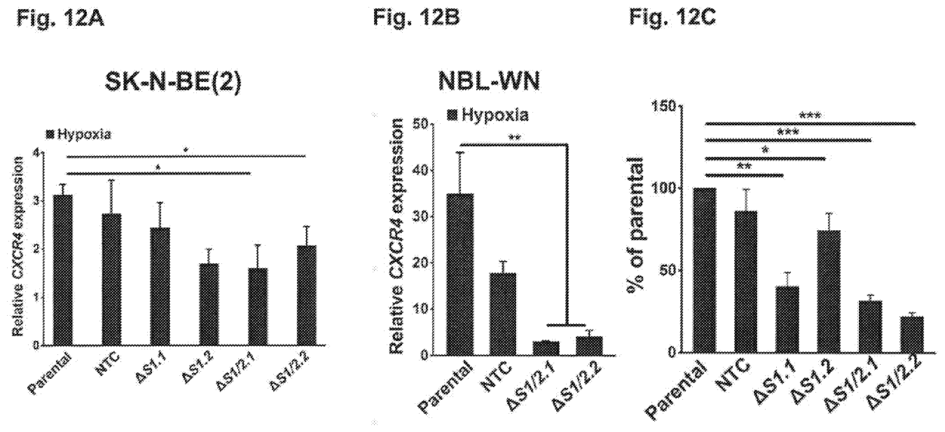

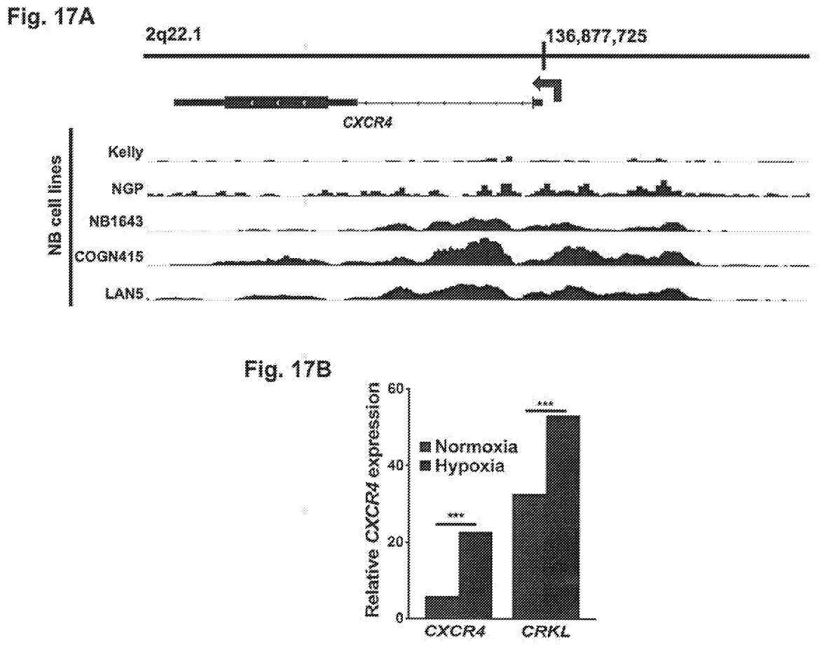

The molecular mechanism behind slow migration was investigated further in ΔS1 and ΔS1/2 cells. Because both cell lines may have an aberrated distribution of 5-hmC, the distribution of 5-hmC in parental SK-N-BE(2) cells was re-examined over time (see FIGS. 1A-1F). Previously identified migration pathways targeted by ‘late’ 5-hmC in hypoxia were utilized to extract a list of genes that were enriched in 5-hmC in hypoxia and common in migration pathways (FIG. 1F). There were two genes that had significantly increased expression in hypoxia: CXCR4 and CRKL (FIGS. 17A-17B). The gene with hypoxic 5-hmC induction and the highest fold change (3.8 fold) in expression was CXCR4 (FIGS. 10E and 17A-17B). CXCR4 encodes a surface receptor that functions in retention of hematopoietic stem cells in the bone marrow and chemotactic guidance in neural progenitor cells. In NB, CXCR4 expression is correlated with metastatic spread and worse outcome. CXCR4 featured 5-hmC enrichment in the gene body and the promoter, indicating 5-hmC augments expression from CXCR4 in hypoxia. Additionally, it was determined that CXCR4 is a direct target of MYCN and HIF-1α in normoxia and hypoxia respectively (FIGS. 10E and 17B). Moreover, CXCR4 is only induced in hypoxia in MYCN amplified cell lines (FIG. 10F). CXCR4 expression was expanded to include RNA-seq data from several NB cell lines with varying MYCN-amplification statuses and MYCN protein levels. CXCR4 was induced in hypoxia in NB lines that had abundant MYCN protein and one with c-MYC protein (SH-SY5Y) (FIG. 10I). In NBL-S cells, CXCR4 was not induced in hypoxia but did have relatively high baseline expression (FIG. 10I). Although not MYCN-amplified, research has shown these cells express MYCN protein, but at lower levels than MYCN-amplified line SK-N-BE(2)-C42. Next, we measured CXCR4 expression in ΔS1/2 SK-N-BE(2) and NBL-WN cells and observed hypoxic CXCR4 was significantly decreased in both ΔS1/2 cell lines (FIGS. 12A-12B). This is possibly because the enrichment of 5-hmC along the promoter or gene body of CXCR4 was altered and therefore CXCR4 expression was no longer augmented by 5-hmC in these cells. The hypoxic induction of CXCR4 was abrogated in ΔS1 or ΔS1/2 cells resulting in decreased migration (FIG. 10G). Further, wild type parental lines SK-N-BE(2) and NBL-WN were treated with CXCR4 antagonist (plerixafor) to determine the effects of CXCR4 antagonism on migration in wild-type cells migration.

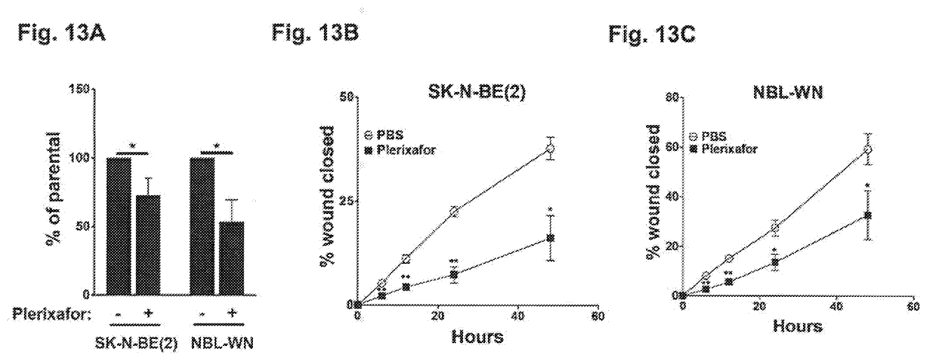

A transwell assay was used to test the cells' ability to migrate in hypoxia. In both cell lines plerixafor-treated cells migrated slower than their control counterparts, indicating that CXCR4 plays a role in NB tumor migration (FIG. 10H). More specifically, to test whether CXCR4 affects NB cell migration, we performed transwell assays with hypoxic SK-N-BE(2) ΔS1/2 cells. It was observed that ΔS1/2 cells migrated slower in hypoxia compared to controls (FIG. 12C). Moreover, ΔS1 cells, which do not exhibit hypoxic induction of TET1 also migrated slower in hypoxia (FIG. 12C). Wound healing assays, a complementary approach, were performed under pseudo-hypoxic conditions and confirmed the migration phenotype in the ΔS1 cells (FIGS. 10A-10B). To test if CXCR4 promotes migration, hypoxic MYCN-amplified NB cell lines SK-N-BE(2) and NBL-WN were treated with plerixafor, a CXCR4 antagonist. In both transwell and wound healing assays, plerixafor-treated cells migrated slower than their control counterparts (FIGS. 13A-13C), indicating CXCR4 directs MYCN-amplified NB tumor migration.

### Example 9: Plerixafor and Neuroblastoma Migration

The direct targeting of MYCN and HIF-1α transcription factors was examined using qPCR to determine their role in neuroblastoma migration. The hypoxic induction of CXCR4 was abrogated in ΔS1 or ΔS1/2 cells as compared to parental SK-N-BE2 CXCR4 expression (FIG. 12A). Similarly, the hypoxic induction of CXCR4 was abrogated in ΔS1/2 cells as compared to parental NBL-WN neuroblastoma cells (FIG. 12B). The addition of plerixafor under hypoxic conditions reduced neuroblastoma migration in SK-N-BE(2) and NBL-WN cells, expressed as percent migration versus parental migration (FIG. 13A). Cell migration, as measured by percent wound closure monitored over 48 hours, was reduced with the addition of plerixafor in SK-N-BE(2) and NBL-WN cells in the presence of hypoxia (FIGS. 13B and 13C).

### Example 10: HIF-1α Promotes TET1 Stability in Hypoxia

To determine if induced TET1 protein levels in hypoxia were a consequence of increased protein stability, protein degradation assay were performed with cycloheximide in normoxic and hypoxic parental SK-N-BE(2) cells. These degradation assays demonstrated that TET1 levels, already very stable in normoxia, persisted even longer in hypoxia (FIG. 8K). To determine if HIF-1α is necessary for the increased stability of TET1 in hypoxia, we performed the same protein degradation assays in normoxia and hypoxia with cells in which HIF1A had been deleted via gene editing (ΔHIF1A) (FIG. 8L). Unlike the parental line, the TET1 stability in hypoxia seemed similar to TET1 stability in normoxia. Half-life calculations in both parental and ΔHIF1A cells in both conditions revealed that normoxic parental and ΔHIF1A SK-N-BE(2) TET1 protein had a half-life of 20 hours, while hypoxic parental SK-N-BE(2) TET1 had a half-life of 40 hours (FIG. 8M). This implies HIF-1α is necessary to stabilize TET1 protein in hypoxia.

Past studies have described TF-TET complexes in which the complex works synergistically to promote TET activity and enhance gene expression. To determine if HIF-1 and TET1 formed part of a complex that could promote stability, we performed co-immunoprecipitation in normoxic and hypoxic SK-N-BE(2) cells. Immunoprecipitation of TET1 also co-immunoprecipitated HIF-1α, indicating HIF-1α and TET1 are part of the same protein complex (FIG. 8N). It was further determined if regulation of TET1 protein activity, independent of TET1 mRNA regulation, was sufficient to induce 5-hmC levels in hypoxia by measuring global hypoxic 5-hmC after inhibition of protein synthesis in both parental and ΔHIF1A SK-N-BE(2) cells. Hypoxic 5-hmC levels continued to increase post-cycloheximide introduction, but hypoxic 5-hmC levels in ΔHIF1A cells were significantly lower than parental hypoxic 5-hmC levels (FIG. 8O). This indicates that the presence of HIF-1α protein augments hypoxic gains of 5-hmC post-protein synthesis inhibition, possibly by augmenting TET1 stability or activity.

To test if the presence of HIF-1α in the cell altered the binding of MYCN to S1 or S2, MYCN ChIP-qPCR was performed in hypoxia to determine if it still bound S1 and/or S2. MYCN bound both S1 and S2 in hypoxia, demonstrating that binding still occurred when HIF-1 was present in the cell (FIG. 8B).

### Summary

The current disclosure demonstrates that in normoxia, TET1 is regulated at two different binding sites by MYCN. Although loss of the first binding site (ΔS1) results in slightly reduced TET1 expression and 5-hmC level, loss of the second (ΔS2) has no significant effect on TET1 or 5-hmC. However, loss of both binding sites resulted in a severe reduction of TET1 expression and around 50% loss of 5-hmC level. In hypoxia, TET1 is regulated by HIF-1 at the exact same binding sites as MYCN. When ΔS1 cells were placed in hypoxia TET1 expression did not change, yet 5-hmC levels did still increase. Hypoxia induction of 5-hmC was only affected in ΔS1/2 cells. Although these cells still featured increases in 5-hmC, they were significantly lower than the hypoxic 5-hmC level of the parental line.

The findings disclosed herein concerning the regulation of the gene CXCR4 in NB cell lines have significant implications for the treatment of MYCN-amplified NB tumors. Herein is disclosed that 5-hmC is integral to other malignant processes such as cell migration. In addition to canonical hypoxic response genes, migration gene CXCR4 is shown to be upregulated under hypoxic conditions across multiple MYCN-amplified cell lines. Yet, when ΔS1/2 cell lines were exposed to hypoxia, CXCR4 expression was no longer induced, and cells migrated slower. Hypoxic upregulation of CXCR4 has implications as a biomarker for aggressive disease and as a therapeutic target in MYCN-amplified NB.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### References

1. Iyer L M, Tahiliani M, Rao A, Aravind L. Prediction of novel families of enzymes involved in oxidative and other complex modifications of bases in nucleic acids. Cell Cycle Georget Tex. 2009;8:1698-710.

2. Tahiliani M, Koh K P, Shen Y, Pastor W A, Bandukwala H, Brudno Y, et al. Conversion of 5-Methylcytosine to 5-Hydroxymethylcytosine in Mammalian DNA by MLL Partner TET1. Science. 2009;324:930-5.

3. Mariani C J, Vasanthakumar A, Madzo J, Yesilkanal A, Bhagat T, Yu Y, et al. TET1-mediated hydroxymethylation facilitates hypoxic gene induction in neuroblastoma. Cell Rep. 2014;7:1343-52.

4. Vasanthakumar A, Godley L A. 5-hydroxymethylcytosine in cancer: significance in diagnosis and therapy. Cancer Genet. 2015;208:167-77.

5. Ficz G, Branco M R, Seisenberger S, Santos F, Krueger F, Hore T A, et al. Dynamic regulation of 5-hydroxymethylcytosine in mouse ES cells and during differentiation. Nature. 2011;473:398-402.

6. Madzo J, Liu H, Rodriguez A, Vasanthakumar A, Sundaravel S, Caces D B D, et al. Hydroxymethylation at Gene Regulatory Regions Directs Stem/Early Progenitor Cell Commitment during Erythropoiesis. Cell Rep. 2014;6:231-44.

7. Laukka T, Mariani C J, Ihantola T, Cao J Z, Hokkanen J, Kaelin W G, et al. Fumarate and Succinate Regulate Expression of Hypoxia-inducible Genes via TET Enzymes. J Biol Chem. 2016;291:4256-65.

8. Kriaucionis S, Heintz N. The nuclear DNA base, 5-hydroxymethylcytosine is present in brain and enriched in Purkinje neurons. Science. 2009;324:929-30.

9. Sanjana N E, Shalem O, Zhang F. Improved vectors and genome-wide libraries for CRISPR screening. Nat Methods. 2014;11:783-4.

10. Durbin A D, Zimmerman M W, Dharia N V, Abraham B J, Iniguez A B, Weichert-Leahey N, et al. Selective gene dependencies in MYCN-amplified neuroblastoma include the core transcriptional regulatory circuitry. Nat Genet. 2018;50:1240-6.

11. Harenza J L, Diamond M A, Adams R N, Song M M, Davidson H L, Hart L S, et al. Transcriptomic profiling of 39 commonly-used neuroblastoma cell lines. Sci Data. 2017;4:170033.

12. Molenaar J J, Koster J, Zwijnenburg D A, van Sluis P, Valentijn L J, van der Ploeg I, et al. Sequencing of neuroblastoma identifies chromothripsis and defects in neuritogenesis genes. Nature. 2012;483:589-93.

13. Thienpont B, Steinbacher J, Zhao H, D'Anna F, Kuchnio A, Ploumakis A, et al. Tumour hypoxia causes DNA hypermethylation by reducing TET activity. Nature. 2016;537:63-8.

14. Zeid R, Lawlor M A, Poon E, Reyes J M, Fulciniti M, Lopez M A, et al. Enhancer invasion shapes MYCN-dependent transcriptional amplification in neuroblastoma. Nat Genet. 2018;50:515-23.

15. Song C-X, Yi C, He C. Mapping new nucleotide variants in the genome and transcriptome. Nat Biotechnol. 2012;30:1107-16.

16. Edsjö A, Holmquist L, Påhlman S. Neuroblastoma as an experimental model for neuronal differentiation and hypoxia-induced tumor cell dedifferentiation. Semin Cancer Biol. 2007;17:248-56.

17. Good C R, Madzo J, Patel B, Maegawa S, Engel N, Jelinek J, et al. A novel isoform of TET1 that lacks a CXXC domain is overexpressed in cancer. Nucleic Acids Res. 2017;45:8269-81.

18. Zhang W, Xia W, Wang Q, Towers A J, Chen J, Gao R, et al. Isoform Switch of TET1 Regulates DNA Demethylation and Mouse Development. Mol Cell. 2016;64:1062-73.

19. Gao T, He B, Liu S, Zhu H, Tan K, Qian J. EnhancerAtlas: a resource for enhancer annotation and analysis in 105 human cell/tissue types. Bioinformatics. 2016;32:3543-51.

20. Lutz W, Stöhr M, Schürmann J, Wenzel A, Löhr A, Schwab M. Conditional expression of N-myc in human neuroblastoma cells increases expression of alpha-prothymosin and ornithine decarboxylase and accelerates progression into S-phase early after mitogenic stimulation of quiescent cells. Oncogene. 1996;13:803-12.

21. Kalatskaya I, Berchiche Y A, Gravel S, Limberg B J, Rosenbaum J S, Heveker N. AMD3100 is a CXCR7 ligand with allosteric agonist properties. Mol Pharmacol. 2009;75:1240-7.

22. Klein S, Abraham M, Bulvik B, Dery E, Weiss I D, Barashi N, et al. CXCR4 Promotes Neuroblastoma Growth and Therapeutic Resistance through miR-15a/16-1-Mediated ERK and BCL2/Cyclin D1 Pathways. Cancer Res. 2018;78:1471-83.

23. Thomas P D, Campbell M J, Kejariwal A, Mi H, Karlak B, Daverman R, et al. PANTHER: A Library of Protein Families and Subfamilies Indexed by Function. Genome Res. 2003;13:2129-41.

1. A method of treating neuroblastoma (NB) in a subject in need thereof, comprising:
a) obtaining a sample of a NB tumor from the subject;
b) measuring MYCN gene expression in the NB tumor sample; and
c) administering a therapeutically effective amount of a CXCR4 antagonist to the patient if MYCN gene amplification is present compared to control.

2. The method of claim 1, wherein the sample is obtained via tumor biopsy.

3. The method of claim 2, wherein the tumor biopsy is a bone marrow biopsy, an endoscopic biopsy, a fine-needle aspiration, a core needle biopsy, a vacuum-assisted biopsy, an image-guided biopsy, a shave biopsy, a punch biopsy, an incisional biopsy, an excisional biopsy, or a surgical biopsy.

4. The method of claim 1, wherein MYCN gene amplification is measured using FISH.

5. The method of claim 1, wherein the CXCR4 antagonist is plerixafor, a T140 analog, BL-8040, TN14003, MSX-122, TG-0054, FC122, FC131, AMD070, an AMD070 derivative, FC131, AMD3465, an AMD3465 analogue, WZ811, MSX122, NB325, NSC56612, KRH-3955, CTCE-9908, POL6326, or combinations thereof.

6. The method of claim 1, wherein the CXCR4 antagonist is administered if MYCN gene amplification is 4-fold or more.

7. The method of claim 6, wherein the neuroblastoma is an MYCN-amplified neuroblastoma.

8. A method of treating neuroblastoma (NB) in a subject in need thereof, comprising:
a) obtaining a sample of a NB tumor from the subject;
b) measuring CXCR4 gene expression in the NB tumor sample; and
c) administering a therapeutically effective amount of a CXCR4 antagonist to the patient if CXCR4 gene expression in the sample is elevated compared to control.

9. The method of claim 8, wherein the CXCR4 antagonist is plerixafor, a T140 analog, BL-8040, TN14003, MSX-122, TG-0054, FC122, FC131, AMD070, an AMD070 derivative, FC131, AMD3465, an AMD3465 analogue, WZ811, MSX122, NB325, NSC56612, KRH-3955, CTCE-9908, POL6326, or combinations thereof.

10. The method of claim 9, wherein the CXCR4 antagonist reduces or prevents NB cell migration.

11. A method of treating a solid tumor in a subject in need thereof, comprising:
a) reducing or preventing tumor cell migration by administering to the subject a therapeutically effective amount of a CXCR4 antagonist; and
b) administering to the subject a therapeutically effective amount of a secondary therapeutic agent.

12. The method of claim 11, wherein the secondary therapeutic agent is a MYCN inhibitor that eliminates or reduces MYCN binding to the superenhancer located in the first intron and/or the second intron of the solid tumor ten-eleven translocation methylcytosine dioxygenase 1 (TET1) gene.

13. The method of claim 11, wherein the secondary therapeutic agent is an antineoplastic agent, hypoxia-inducing factor-1α, hypoxia-inducing factor-1β inhibitor, an inhibitor that binds or reduces the superenhancer located in TET1 intron 1 (S1) an inhibitor that binds or reduces the superenhancer located in the second intron of TET 1 (S2), or a MYCN inhibitor.

14. The method of claim 13, wherein the secondary therapeutic agent is a hypoxia-inducible factor (HIF) inhibitor.

15. The method of claim 14, wherein the HIF1 inhibitor is Roxadustat, Bortezomib, Romidespin, Temsirolimus, Perifosine, 2-methoxyestradiol, Echinomycin, Geldanamycin, 17-AAG, 17-DMAG, or MK-6482.

16. The method of claim 15, wherein the HIF-1 inhibitor eliminates or reduces HIF-1α function in the solid tumor.

17. The method of claim 15, wherein the HIF-1 inhibitor eliminates or reduces HIF-1β function in the solid tumor.

18. The method of claim 11, wherein the CXCR4 antagonist and/or secondary therapeutic agent is administered to the subject orally and/or intravenously.

19. A method of treating neuroblastoma (NB) in a subject in need thereof, comprising:
a) obtaining a sample of an NB via tumor biopsy from the subject;
b) determining cell surface CXCR4 expression level in the NB tumor sample; and
c) administering to the subject a therapeutically effective amount of a CXCR4 antagonist to the patient if the cell surface CXCR 4 expression level is elevated compared to control.


SEQUENCE LISTING (15 sequences)

SEQ ID NO: 1 - DNA, 30 bp, Artificial Sequence
  Synthetic Oligonucleotide
gagccgggccccacgtggaacgcgccggcg

SEQ ID NO: 2 - DNA, 9 bp, Artificial Sequence
  Synthetic Oligonucleotide
gagccggcg

SEQ ID NO: 3 - DNA, 15 bp, Artificial Sequence
  Synthetic Oligonucleotide
gagcccggcccggcg

SEQ ID NO: 4 - DNA, 32 bp, Artificial Sequence
  Synthetic Oligonucleotide
ctggtcgtgcagcacgtgaggggcctggtcct

SEQ ID NO: 5 - DNA, 30 bp, Artificial Sequence
  Synthetic Oligonucleotide
ctggtcgtgcagcatgaggggcctggtcct

SEQ ID NO: 6 - DNA, 30 bp, Artificial Sequence; identical to SEQ ID NO: 5
  Synthetic Oligonucleotide

SEQ ID NO: 7 - DNA, 30 bp, Artificial Sequence
  Synthetic Oligonucleotide
tcttggcatgcccagcacgtctacagtgct

SEQ ID NO: 8 - DNA, 10 bp, Artificial Sequence
  Synthetic Oligonucleotide
tctgcatgct

SEQ ID NO: 9 - DNA, 7 bp, Artificial Sequence
  Synthetic Oligonucleotide
tcttgct

SEQ ID NO: 10 - DNA, 32 bp, Artificial Sequence; identical to SEQ ID NO: 4
  Synthetic Oligonucleotide

SEQ ID NO: 11 - DNA, 30 bp, Artificial Sequence; identical to SEQ ID NO: 5
  Synthetic Oligonucleotide

SEQ ID NO: 12 - DNA, 30 bp, Artificial Sequence; identical to SEQ ID NO: 5
  Synthetic Oligonucleotide

SEQ ID NO: 13 - DNA, 30 bp, Artificial Sequence; identical to SEQ ID NO: 7
  Synthetic Oligonucleotide

SEQ ID NO: 14 - DNA, 10 bp, Artificial Sequence
  Synthetic Oligonucleotide
tctgcatgct

SEQ ID NO: 15 - DNA, 7 bp, Artificial Sequence
  Synthetic Oligonucleotide
tcttgct